(19) United States
(12) Patent Application Publication
(10) Pub. No.: US 2026/0053582 A1
(43) Pub. Date: Feb. 26, 2026

(54) GRAVITY COMPENSATION OF END EFFECTOR ARM FOR ROBOTIC SURGICAL SYSTEM

(71) Applicant: GLOBUS MEDICAL, INC., AUDUBON, PA (US)

(72) Inventors: Olivier Chappuis, Lutry (CH); Szymon Kostrzewski, Lausanne (CH)

(21) Appl. No.: 19/377,133

(22) Filed: Nov. 3, 2025

Related U.S. Application Data
(63) Continuation of application No. 17/806,745, filed on Jun. 14, 2022, now Pat. No. 12,458,454.
(60) Provisional application No. 63/212,949, filed on Jun. 21, 2021.

(51) Int. Cl. A61B 34/30; A61B 34/00

(52) U.S. Cl. CPC A61B 34/30; A61B 34/70; A61B 34/25

(57) ABSTRACT

An end effector arm for use with a surgical navigation system includes a base configured to attach to an end effector coupler of a surgical robot arm and a mechanical linkage. The mechanical linkage includes a first end rotatably coupled to the base and a second end opposite the first end, the second end configured to be removably coupled to a handheld surgical tool. The end effector arm further includes a spring mechanism configured to impart a variable rotational force on the mechanical linkage based on an angle of rotation of the mechanical linkage with respect to the base.

### CROSS-REFERENCE TO RELATED APPLICATIONS

This application is a continuation of U.S. patent application Ser. No. 17/806,745, filed on Jun. 14, 2022, which claims the benefit of priority to U.S. Provisional Patent Application No. 63/212,949, filed Jun. 21, 2021, the contents of which are incorporated by reference herein in their entirety.

This application is related to, but does not claim priority to U.S. patent application Ser. No. 16/587,203, filed Sep. 30, 2019, and U.S. patent application Ser. No. 16/737,054, filed Jan. 8, 2020, the contents of which are incorporated by reference herein in their entirety.

### TECHNICAL FIELD

The present invention relates to robotic surgery and more particularly to an end effector arm for use with a robot assisted surgical system.

### BACKGROUND

There are a number of surgical interventions requiring osteotomy, i.e. cutting an anatomical structure such as a bone along a target plane. Total knee arthroplasty typically requires cutting both the femoral epiphysis and tibial epiphysis in order to remove the damaged bone and cartilage and install a knee prosthesis. A surgeon may perform five or more cuts on the femur and one or more cuts on the tibia using an oscillating surgical saw.

During orthopedic surgeries, including joints and knees, it is important to accurately align and stabilize the saw while cutting a desired location on a bone. The surgeon's limited visibility to the surgical site combined with the difficultly in controlling movement of the saw creates a risk that an undesired part of a bone or adjacent tissue becomes cut. Vibrations generated by the saw while cutting can reduce the accuracy of the cuts. During knee surgery, the precision of a bone cut (planar cuts) affects how precisely the implant can be connected to the exposed bone.

In some conventional systems, a direct sagittal saw blade guidance structure may use a passive kinematics positioned in the space by a robotic arm, which to constrain the blade in its resection plane. The passive structure, further designated as an end effector arm, may include a three linkage serial structure offering 3 degrees of freedom (two translations and one rotation) to the blade. A sagittal saw handpiece is rigidly connected to the passive structure through the blade and provide mechanical power to actuate the blade. In order to achieve sufficient resection accuracy, the passive structure has to be designed with the highest possible transverse stiffness. Integration of measuring function by means of encoders, as well as manufacturing cost reduction lead to the selection of Aluminum as appropriate material for the structural parts of the link. This impacts the weight of these structural parts. When the resection plane is horizontal, the gravity force due to links mass is entirely supported by the joints bearings. However, in many resections, the resection plane is inclined with respect to the horizontal plane or almost vertical. In such configurations, the weight of links is partially or totally supported by surgeon's hand, which may cause undesirable strain or stress on the surgeon's hand and which may negatively impact the surgeon's performance.

Therefore, there is a need for a surgical guidance system that allows for freedom of movement of a saw blade or other handheld surgical tool without requiring the surgeon to support the weight of the additional system components.

### SUMMARY

The present invention relates to robotic surgery and more particularly to an end effector arm for use with a robot assisted surgical system.

According to some embodiments, am end effector arm for use with a surgical navigation system includes a base configured to attach to an end effector coupler of a surgical robot arm and a mechanical linkage. The mechanical linkage includes a first end rotatably coupled to the base and a second end opposite the first end, the second end configured to be removably coupled to a handheld surgical tool. The end effector arm further includes a spring mechanism configured to impart a variable rotational force on the mechanical linkage based on an angle of rotation of the mechanical linkage with respect to the base.

According to some embodiment, a surgical tool guidance system includes a robot arm configured to be positioned by a surgical robot including an end effector coupler. The system further includes an end effector arm including a base configured to attach to the end effector coupler of the robot arm and a mechanical linkage. The mechanical linkage includes a first end rotatably coupled to the base and a second end opposite the first end. The end effector arm further includes a spring mechanism configured to impart a variable rotational force on the mechanical linkage based on an angle of rotation of the mechanical linkage with respect to the base. The system further includes a handheld surgical tool configured to couple to the second end of the mechanical linkage.

According to some embodiments, a surgical system includes a tracking system configured to determine a pose of an anatomical structure that is to be engaged by a handheld surgical tool and to determine a pose of the surgical tool. The system further includes a surgical robot including a robot base, and a robot arm connected to the robot base, the robot arm comprising an end effector coupler. The robot further includes at least one motor operatively connected to move the robot arm relative to the robot base. The system further includes an end effector arm including a base configured to attach to the end effector coupler of the robot arm and a mechanical linkage. The mechanical linkage includes a first end rotatably coupled to the base and a second end opposite the first end. The end effector arm further includes a spring mechanism configured to impart a variable rotational force on the mechanical linkage based on an angle of rotation of the mechanical linkage with respect to the base. The system further includes a handheld surgical tool coupled to the second end of the mechanical linkage.

### BRIEF DESCRIPTION OF THE DRAWINGS

The accompanying drawings, which are included to provide a further understanding of the disclosure and are incorporated in a constitute a part of this application, illustrate certain non-limiting embodiments of inventive concepts. In the drawings:

FIG. 1 illustrates an embodiment of a surgical system according to some embodiments of the present disclosure;

FIG. 2 illustrates a surgical robot component of the surgical system of FIG. 1 according to some embodiments of the present disclosure;

FIG. 3 illustrates a camera tracking system component of the surgical system of FIG. 1 according to some embodiments of the present disclosure;

FIG. 4 illustrates an embodiment of a passive end effector that is connectable to a robot arm and configured according to some embodiments of the present disclosure;

FIG. 5 illustrates a medical operation in which a surgical robot and a camera system are disposed around a patient;

FIG. 6 illustrates an embodiment of an end effector coupler of a robot arm configured for connection to a passive end effector according to some embodiments of the present disclosure;

FIG. 7 illustrates an embodiment of a cut away of the end effector coupler of FIG. 6;

FIG. 8 illustrates a block diagram of components of a surgical system according to some embodiments of the present disclosure;

FIG. 9 illustrates a block diagram of a surgical system computer platform that includes a surgical planning computer which may be separate from and operationally connected to a surgical robot or at least partially incorporated therein according to some embodiments of the present disclosure;

FIG. 10 illustrates an embodiment of a C-Arm imaging device that can be used in combination with the surgical robot and passive end effector in accordance with some embodiments of the present disclosure;

FIG. 11 illustrates an embodiment of an O-Arm imaging device that can be used in combination with the surgical robot and passive end effector in accordance with some embodiments of the present disclosure; and

FIG. 12 illustrate an embodiment of a passive end effector configured in accordance with some embodiments of the present disclosure.

FIG. 13 is a screenshot of a display showing the progress of bone cuts during a surgical procedure.

FIG. 14 illustrates an embodiment of an end effector arm having a spring mechanism for compensating for gravitational force on the end effector arm components, in accordance with some embodiments of the present disclosure.

FIG. 15 illustrates an embodiment of the end effector arm of FIG. 14 having a spring mechanism illustrating forces applied to the end effector arm components when the end effector arm is in a vertical resection plane, according to some embodiments of the present disclosure.

FIGS. 16A and 16B illustrate a portion of the end effector arm of FIGS. 14 and 15 illustrating forces applied to a first link of the end effector arm when the end effector arm is in a docked position and in an extended position, according to some embodiments of the present disclosure.

FIG. 17 is a graph of the gravitational force and the compensation force on an end effector arm when the end effector arm is in a vertical resection plane, according to some embodiments of the present disclosure.

FIG. 18 is a graph of the gravitational force and the compensation force on the end effector arm when the end effector arm is in a resection plane of approximately 30 degrees with respect to horizontal, according to some embodiments of the present disclosure.

FIGS. 19A-19C illustrate a locking mechanism configured to selectively fix a surgical tool with respect to a base of an end effector arm, according to some embodiments of the present disclosure.

### DETAILED DESCRIPTION

Inventive concepts will now be described more fully hereinafter with reference to the accompanying drawings, in which examples of embodiments of inventive concepts are shown. Inventive concepts may, however, be embodied in many different forms and should not be construed as limited to the embodiments set forth herein. Rather, these embodiments are provided so that this disclosure will be thorough and complete, and will fully convey the scope of various present inventive concepts to those skilled in the art. It should also be noted that these embodiments are not mutually exclusive. Components from one embodiment may be tacitly assumed to be present or used in another embodiment.

Various embodiments disclosed herein are directed to improvements in operation of a surgical system when performing surgical interventions requiring osteotomy. Passive end effectors are disclosed that are connectable to a robot arm positioned by a surgical robot. The passive end effectors have mechanisms that constrain movement of a tool attachment mechanism, such as a surgical saw, to a range of movement. The mechanisms may be configured to constrain a cutting plane of the saw blade to be parallel to the working plane.

The passive end effectors includes a spring mechanism configured to impart a variable rotational force on the mechanical linkage based on an angle of rotation of the mechanical linkage with respect to the base. The variable rotational force may be sufficient to overcome a gravitational force on a distal end of the end effector arm through a range of angles of rotation when the end effector is in an inclined and/or vertical resection plane.

These and other related embodiments can operate to improve the precision of the guidance of the tool compared to other robotic and manual solutions for surgeries and reduce the effort and fatigue on a surgeon's hand due to the weight of the end effector arm. The spring mechanism may also be configured to minimize variation of the compensation effect across the entire range of motion of the end effector arm to provide gradual and predictable changes in net forces on the end effector.

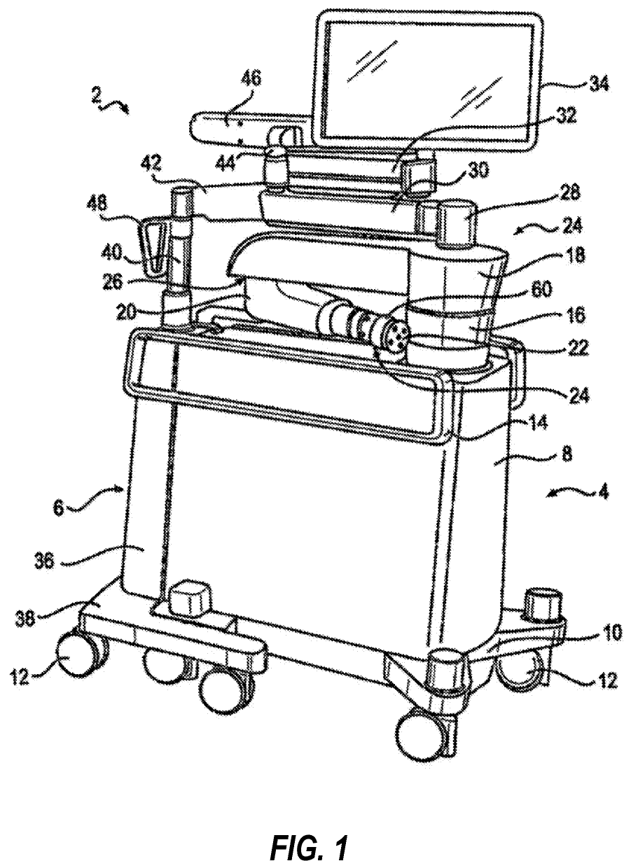

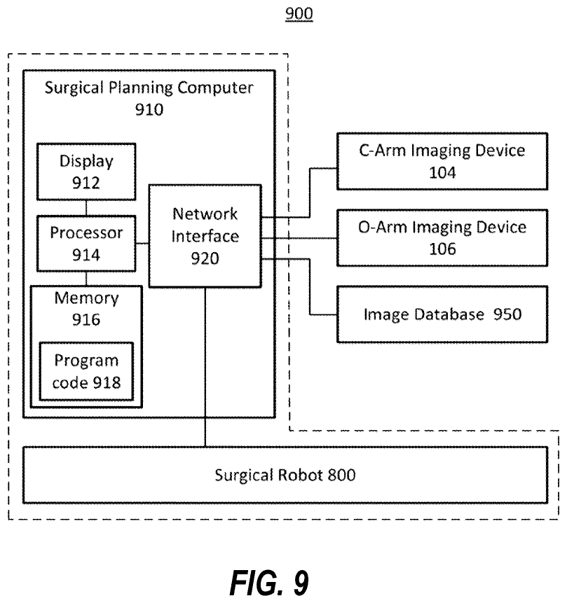

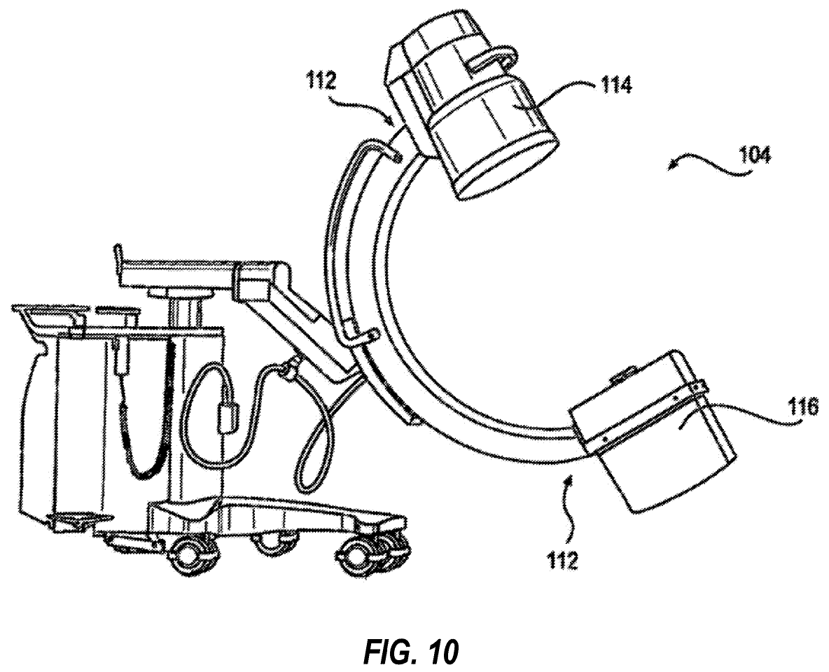

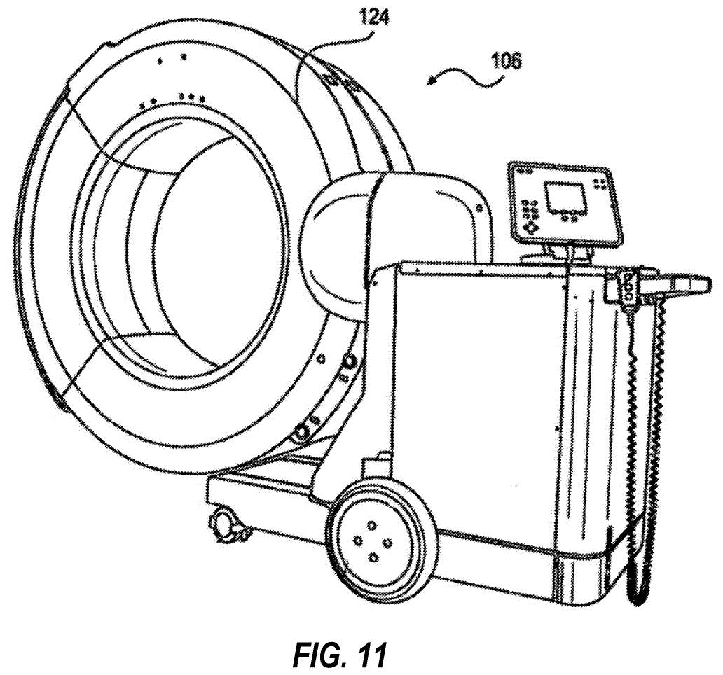

FIG. 1 illustrates an embodiment of a surgical system 2 according to some embodiments of the present disclosure. Prior to performance of an orthopedic surgical procedure, a three-dimensional (“3D”) image scan may be taken of a planned surgical area of a patient using, e.g., the C-Arm imaging device 104 of FIG. 10 or O-Arm imaging device 106 of FIG. 11, or from another medical imaging device such as a computed tomography (CT) image or Mill. This scan can be taken pre-operatively (e.g. few weeks before procedure, most common) or intra-operatively. However, any known 3D or 2D image scan may be used in accordance with various embodiments of the surgical system 2. The image scan is sent to a computer platform in communication with the surgical system 2, such as the surgical system computer platform 900 of FIG. 9 which includes the surgical robot 800 (e.g., the robot of surgical system 2 in FIG. 1) and a surgical planning computer 910. A surgeon reviewing the image scan(s) on a display device of the surgical planning computer 910 (FIG. 9) generates a surgical plan defining a target plane where an anatomical structure of the patient is to be cut. This plane is a function of patient anatomy constraints, selected implant and its size. In some embodiments, the surgical plan defining the target plane is planned on the 3D image scan displayed on a display device.

The surgical system 2 of FIG. 1 can assist surgeons during medical procedures by, for example, holding tools, aligning tools, using tools, guiding tools, and/or positioning tools for use. In some embodiments, surgical system 2 includes a surgical robot 4 and a camera tracking system 6. Both systems may be mechanically coupled together by any various mechanisms. Suitable mechanisms can include, but are not limited to, mechanical latches, ties, clamps, or buttresses, or magnetic or magnetized surfaces. The ability to mechanically couple surgical robot 4 and camera tracking system 6 can allow for surgical system 2 to maneuver and move as a single unit, and allow surgical system 2 to have a small footprint in an area, allow easier movement through narrow passages and around turns, and allow storage within a smaller area.

An orthopedic surgical procedure may begin with the surgical system 2 moving from medical storage to a medical procedure room. The surgical system 2 may be maneuvered through doorways, halls, and elevators to reach a medical procedure room. Within the room, the surgical system 2 may be physically separated into two separate and distinct systems, the surgical robot 4 and the camera tracking system 6. Surgical robot 4 may be positioned adjacent the patient at any suitable location to properly assist medical personnel. Camera tracking system 6 may be positioned at the base of the patient, at patient shoulders or any other location suitable to track the present pose and movement of the pose of tracks portions of the surgical robot 4 and the patient. Surgical robot 4 and camera tracking system 6 may be powered by an onboard power source and/or plugged into an external wall outlet.

Surgical robot 4 may be used to assist a surgeon by holding and/or using tools during a medical procedure. To properly utilize and hold tools, surgical robot 4 may rely on a plurality of motors, computers, and/or actuators to function properly. Illustrated in FIG. 1, robot body 8 may act as the structure in which the plurality of motors, computers, and/or actuators may be secured within surgical robot 4. Robot body 8 may also provide support for robot telescoping support arm 16. In some embodiments, robot body 8 may be made of any suitable material. Suitable material may be, but is not limited to, metal such as titanium, aluminum, or stainless steel, carbon fiber, fiberglass, or heavy-duty plastic. The size of robot body 8 may provide a solid platform supporting attached components, and may house, conceal, and protect the plurality of motors, computers, and/or actuators that may operate attached components.

Robot base 10 may act as a lower support for surgical robot 4. In some embodiments, robot base 10 may support robot body 8 and may attach robot body 8 to a plurality of powered wheels 12. This attachment to wheels may allow robot body 8 to move in space efficiently. Robot base 10 may run the length and width of robot body 8. Robot base 10 may be about two inches to about 10 inches tall. Robot base 10 may be made of any suitable material. Suitable material may be, but is not limited to, metal such as titanium, aluminum, or stainless steel, carbon fiber, fiberglass, or heavy-duty plastic or resin. Robot base 10 may cover, protect, and support powered wheels 12.

In some embodiments, as illustrated in FIG. 1, at least one powered wheel 12 may be attached to robot base 10. Powered wheels 12 may attach to robot base 10 at any location. Each individual powered wheel 12 may rotate about a vertical axis in any direction. A motor may be disposed above, within, or adjacent to powered wheel 12. This motor may allow for surgical system 2 to maneuver into any location and stabilize and/or level surgical system 2. A rod, located within or adjacent to powered wheel 12, may be pressed into a surface by the motor. The rod, not pictured, may be made of any suitable metal to lift surgical system 2. Suitable metal may be, but is not limited to, stainless steel, aluminum, or titanium. Additionally, the rod may comprise at the contact-surface-side end a buffer, not pictured, which may prevent the rod from slipping and/or create a suitable contact surface. The material may be any suitable material to act as a buffer. Suitable material may be, but is not limited to, a plastic, neoprene, rubber, or textured metal. The rod may lift powered wheel 12, which may lift surgical system 2, to any height required to level or otherwise fix the orientation of the surgical system 2 in relation to a patient. The weight of surgical system 2, supported through small contact areas by the rod on each wheel, prevents surgical system 2 from moving during a medical procedure. This rigid positioning may prevent objects and/or people from moving surgical system 2 by accident.

Moving surgical system 2 may be facilitated using robot railing 14. Robot railing 14 provides a person with the ability to move surgical system 2 without grasping robot body 8. As illustrated in FIG. 1, robot railing 14 may run the length of robot body 8, shorter than robot body 8, and/or may run longer the length of robot body 8. Robot railing 14 may be made of any suitable material. Suitable material may be, but is not limited to, metal such as titanium, aluminum, or stainless steel, carbon fiber, fiberglass, or heavy-duty plastic. Robot railing 14 may further provide protection to robot body 8, preventing objects and or personnel from touching, hitting, or bumping into robot body 8.

Robot body 8 may provide support for a Selective Compliance Articulated Robot Arm, hereafter referred to as a “SCARA.” A SCARA 24 may be beneficial to use within the surgical system 2 due to the repeatability and compactness of the robotic arm. The compactness of a SCARA may provide additional space within a medical procedure, which may allow medical professionals to perform medical procedures free of excess clutter and confining areas. SCARA 24 may comprise robot telescoping support 16, robot support arm 18, and/or robot arm 20. Robot telescoping support 16 may be disposed along robot body 8. As illustrated in FIG. 1, robot telescoping support 16 may provide support for the SCARA 24 and display 34. In some embodiments, robot telescoping support 16 may extend and contract in a vertical direction. Robot telescoping support 16 may be made of any suitable material. Suitable material may be, but is not limited to, metal such as titanium or stainless steel, carbon fiber, fiberglass, or heavy-duty plastic. The body of robot telescoping support 16 may be any width and/or height in which to support the stress and weight placed upon it.

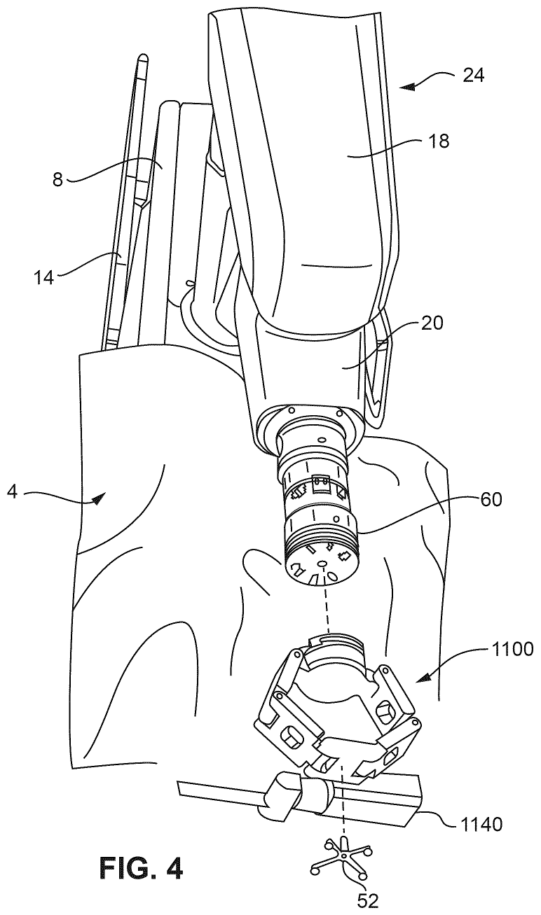

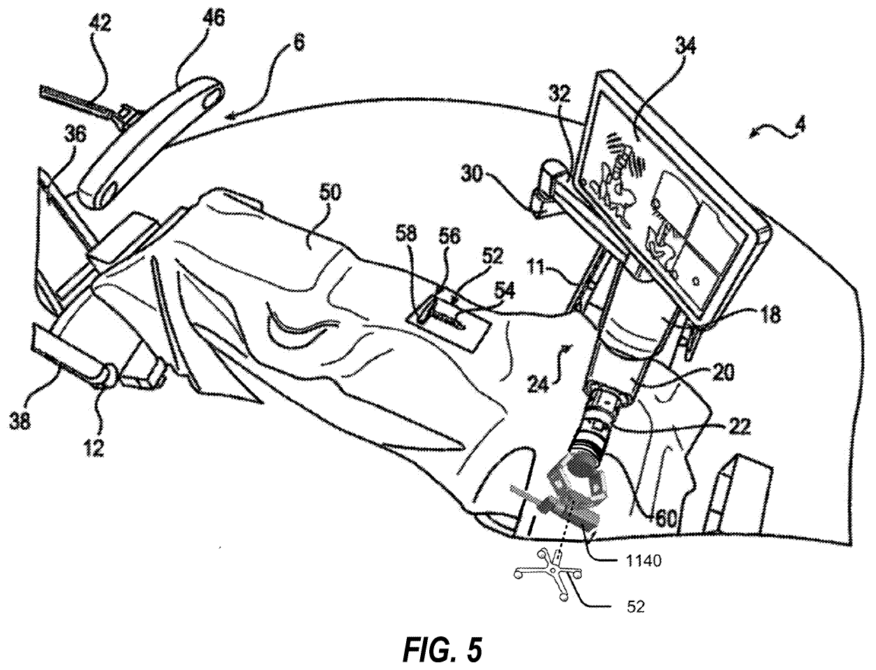

In some embodiments, medical personnel may move SCARA 24 through a command submitted by the medical personnel. The command may originate from input received on display 34 and/or a tablet. The command may come from the depression of a switch and/or the depression of a plurality of switches. Best illustrated in FIGS. 4 and 5, an activation assembly 60 may include a switch and/or a plurality of switches. The activation assembly 60 may be operable to transmit a move command to the SCARA 24 allowing an operator to manually manipulate the SCARA 24. When the switch, or plurality of switches, is depressed the medical personnel may have the ability to move SCARA 24 easily. Additionally, when the SCARA 24 is not receiving a command to move, the SCARA 24 may lock in place to prevent accidental movement by personnel and/or other objects. By locking in place, the SCARA 24 provides a solid platform upon which a passive end effector 1100 and connected surgical saw 1140, shown in FIGS. 4 and 5, are ready for use in a medical operation.

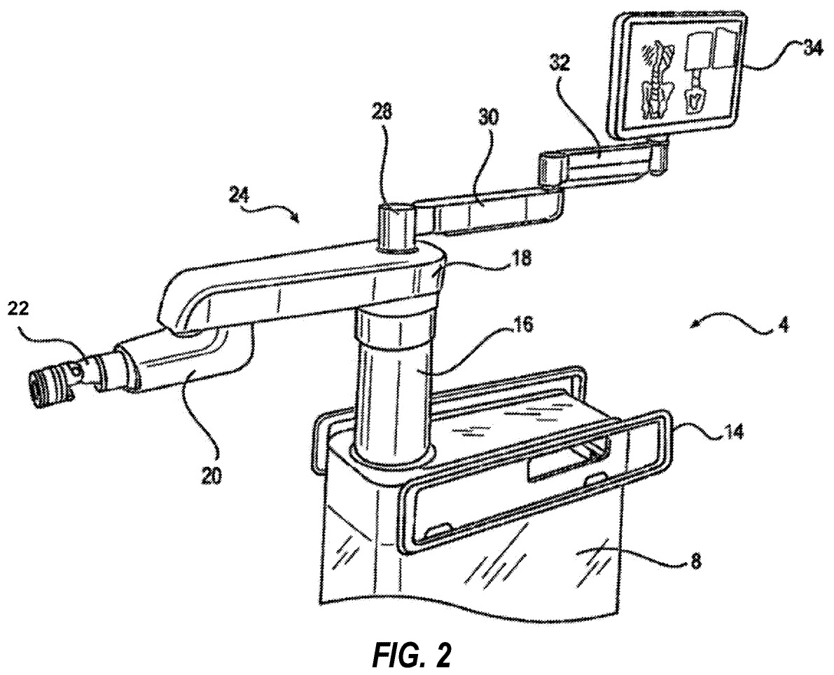

Robot support arm 18 may be disposed on robot telescoping support 16 by various mechanisms. In some embodiments, best seen in FIGS. 1 and 2, robot support arm 18 rotates in any direction in regard to robot telescoping support 16. Robot support arm 18 may rotate three hundred and sixty degrees around robot telescoping support 16. Robot arm 20 may connect to robot support arm 18 at any suitable location. Robot arm 20 may attach to robot support arm 16 by various mechanisms. Suitable mechanisms may be, but is not limited to, nuts and bolts, ball and socket fitting, press fitting, weld, adhesion, screws, rivets, clamps, latches, and/or any combination thereof. Robot arm 20 may rotate in any direction in regards to robot support arm 18, in embodiments, robot arm 20 may rotate three hundred and sixty degrees in regards to robot support arm 18. This free rotation may allow an operator to position robot arm 20 as planned.

The passive end effector 1100 in FIGS. 4 and 5 may attach to robot arm 20 in any suitable location. As will be explained in further detail below, the passive end effector 1100 includes a base, a first mechanism, and a second mechanism. The base is configured to attach to an end effector coupler 22 of the robot arm 20 positioned by the surgical robot 4. Various mechanisms by which the base can attach to the end effector coupler 22 can include, but are not limited to, latch, clamp, nuts and bolts, ball and socket fitting, press fitting, weld, adhesion, screws, rivets, and/or any combination thereof. The first mechanism extends between a rotatable connection to the base and a rotatable connection to a tool attachment mechanism. The second mechanism extends between a rotatable connection to the base and a rotatable connection to the tool attachment mechanism. The first and second mechanisms pivot about the rotatable connections, and may be configured to constrain movement of the tool attachment mechanism to a range of movement within a working plane. The rotatable connections may be pivot joints allowing 1 degree-of-freedom (DOF) motion, universal joints allowing 2 DOF motions, or ball joints allowing 3 DOF motions. The tool attachment mechanism is configured to connect to a surgical saw 1140 having a saw blade or saw blade directly. The surgical saw 1140 may be configured to oscillate the saw blade for cutting. The first and second mechanisms may be configured to constrain a cutting plane of the saw blade to be parallel to the working plane. Pivot joints may be preferably used for connecting the planar mechanisms when the passive end effector is to be configured to constrain motion of the saw blade to the cutting plane.

The tool attachment mechanism may connect to the surgical saw 1140 or saw blade through various mechanisms that can include, but are not limited to, a screw, nut and bolt, clamp, latch, tie, press fit, or magnet. In some embodiments, a dynamic reference array 52 is attached to the passive end effector 1100, e.g., to the tool attachment mechanism, and/or is attached to the surgical saw 1140. Dynamic reference arrays, also referred to as “DRAs” herein, are rigid bodies which may be disposed on a patient, the surgical robot, the passive end effector, and/or the surgical saw in a navigated surgical procedure. The camera tracking system 6 or other 3D localization system is configured to track in real-time the pose (e.g., positions and rotational orientations) of tracking markers of the DRA. The tracking markers may include the illustrated arrangement of balls or other optical markers. This tracking of 3D coordinates of tracking markers can allow the surgical system 2 to determine the pose of the DRA 52 in any space in relation to the target anatomical structure of the patient 50 in FIG. 5.

As illustrated in FIG. 1, a light indicator 28 may be positioned on top of the SCARA 24. Light indicator 28 may illuminate as any type of light to indicate “conditions” in which surgical system 2 is currently operating. For example, the illumination of green may indicate that all systems are normal. Illuminating red may indicate that surgical system 2 is not operating normally. A pulsating light may mean surgical system 2 is performing a function. Combinations of light and pulsation may create a nearly limitless amount of combinations in which to communicate the current operating conditions, states, or other operational indications. In some embodiments, the light may be produced by LED bulbs, which may form a ring around light indicator 28. Light indicator 28 may comprise a fully permeable material that may let light shine through the entirety of light indicator 28.

Light indicator 28 may be attached to lower display support 30. Lower display support 30, as illustrated in FIG. 2 may allow an operator to maneuver display 34 to any suitable location. Lower display support 30 may attach to light indicator 28 by any suitable mechanism. In embodiments, lower display support 30 may rotate about light indicator 28. In embodiments, lower display support 30 may attach rigidly to light indicator 28. Light indicator 28 may then rotate three hundred and sixty degrees about robot support arm 18. Lower display support 30 may be of any suitable length, a suitable length may be about eight inches to about thirty four inches. Lower display support 30 may act as a base for upper display support 32.

Upper display support 32 may attach to lower display support 30 by any suitable mechanism. Upper display support 32 may be of any suitable length, a suitable length may be about eight inches to about thirty four inches. In embodiments, as illustrated in FIG. 1, upper display support 32 may allow display 34 to rotate three hundred and sixty degrees in relation to upper display support 32. Likewise, upper display support 32 may rotate three hundred and sixty degrees in relation to lower display support 30.

Display 34 may be any device which may be supported by upper display support 32. In embodiments, as illustrated in FIG. 2, display 34 may produce color and/or black and white images. The width of display 34 may be about eight inches to about thirty inches wide. The height of display 34 may be about six inches to about twenty two inches tall. The depth of display 34 may be about one-half inch to about four inches.

In embodiments, a tablet may be used in conjunction with display 34 and/or without display 34. In embodiments, the table may be disposed on upper display support 32, in place of display 34, and may be removable from upper display support 32 during a medical operation. In addition the tablet may communicate with display 34. The tablet may be able to connect to surgical robot 4 by any suitable wireless and/or wired connection. In some embodiments, the tablet may be able to program and/or control surgical system 2 during a medical operation. When controlling surgical system 2 with the tablet, all input and output commands may be duplicated on display 34. The use of a tablet may allow an operator to manipulate surgical robot 4 without having to move around patient 50 and/or to surgical robot 4.

As illustrated in FIG. 5, camera tracking system 6 works in conjunction with surgical robot 4 through wired or wireless communication networks. Referring to FIGS. 1 and 5, camera tracking system 6 can include some similar components to the surgical robot 4. For example, camera body 36 may provide the functionality found in robot body 8. Robot body 8 may provide the structure upon which camera 46 is mounted. The structure within robot body 8 may also provide support for the electronics, communication devices, and power supplies used to operate camera tracking system 6. Camera body 36 may be made of the same material as robot body 8. Camera tracking system 6 may communicate directly to the tablet and/or display 34 by a wireless and/or wired network to enable the tablet and/or display 34 to control the functions of camera tracking system 6.

Camera body 36 is supported by camera base 38. Camera base 38 may function as robot base 10. In the embodiment of FIG. 1, camera base 38 may be wider than robot base 10. The width of camera base 38 may allow for camera tracking system 6 to connect with surgical robot 4. As illustrated in FIG. 1, the width of camera base 38 may be large enough to fit outside robot base 10. When camera tracking system 6 and surgical robot 4 are connected, the additional width of camera base 38 may allow surgical system 2 additional maneuverability and support for surgical system 2.

As with robot base 10, a plurality of powered wheels 12 may attach to camera base 38. Powered wheel 12 may allow camera tracking system 6 to stabilize and level or set fixed orientation in regards to patient 50, similar to the operation of robot base 10 and powered wheels 12. This stabilization may prevent camera tracking system 6 from moving during a medical procedure and may keep camera 46 from losing track of one or more DRAs 52 connected to an anatomical structure 54 and/or tool 58 within a designated area 56 as shown in FIG. 5. This stability and maintenance of tracking enhances the ability of surgical robot 4 to operate effectively with camera tracking system 6. Additionally, the wide camera base 38 may provide additional support to camera tracking system 6. Specifically, a wide camera base 38 may prevent camera tracking system 6 from tipping over when camera 46 is disposed over a patient, as illustrated in FIG. 5. Without the wide camera base 38, the outstretched camera 46 may unbalance camera tracking system 6, which may result in camera tracking system 6 falling over.

Camera telescoping support 40 may support camera 46. In embodiments, telescoping support 40 may move camera 46 higher or lower in the vertical direction. Telescoping support 40 may be made of any suitable material in which to support camera 46. Suitable material may be, but is not limited to, metal such as titanium, aluminum, or stainless steel, carbon fiber, fiberglass, or heavy-duty plastic. Camera handle 48 may be attached to camera telescoping support 40 at any suitable location. Cameral handle 48 may be any suitable handle configuration. A suitable configuration may be, but is not limited to, a bar, circular, triangular, square, and/or any combination thereof. As illustrated in FIG. 1, camera handle 48 may be triangular, allowing an operator to move camera tracking system 6 into a planned position before a medical operation. In embodiments, camera handle 48 may be used to lower and raise camera telescoping support 40. Camera handle 48 may perform the raising and lowering of camera telescoping support 40 through the depression of a button, switch, lever, and/or any combination thereof.

Lower camera support arm 42 may attach to camera telescoping support 40 at any suitable location, in embodiments, as illustrated in FIG. 1, lower camera support arm 42 may rotate three hundred and sixty degrees around telescoping support 40. This free rotation may allow an operator to position camera 46 in any suitable location. Lower camera support arm 42 may be made of any suitable material in which to support camera 46. Suitable material may be, but is not limited to, metal such as titanium, aluminum, or stainless steel, carbon fiber, fiberglass, or heavy-duty plastic. Cross-section of lower camera support arm 42 may be any suitable shape. Suitable cross-sectional shape may be, but is not limited to, circle, square, rectangle, hexagon, octagon, or i-beam. The cross-sectional length and width may be about one to ten inches. Length of the lower camera support arm may be about four inches to about thirty-six inches. Lower camera support arm 42 may connect to telescoping support 40 by any suitable mechanism. Suitable mechanism may be, but is not limited to, nuts and bolts, ball and socket fitting, press fitting, weld, adhesion, screws, rivets, clamps, latches, and/or any combination thereof. Lower camera support arm 42 may be used to provide support for camera 46. Camera 46 may be attached to lower camera support arm 42 by any suitable mechanism. Suitable mechanism may be, but is not limited to, nuts and bolts, ball and socket fitting, press fitting, weld, adhesion, screws, rivets, and/or any combination thereof. Camera 46 may pivot in any direction at the attachment area between camera 46 and lower camera support arm 42. In embodiments a curved rail 44 may be disposed on lower camera support arm 42.

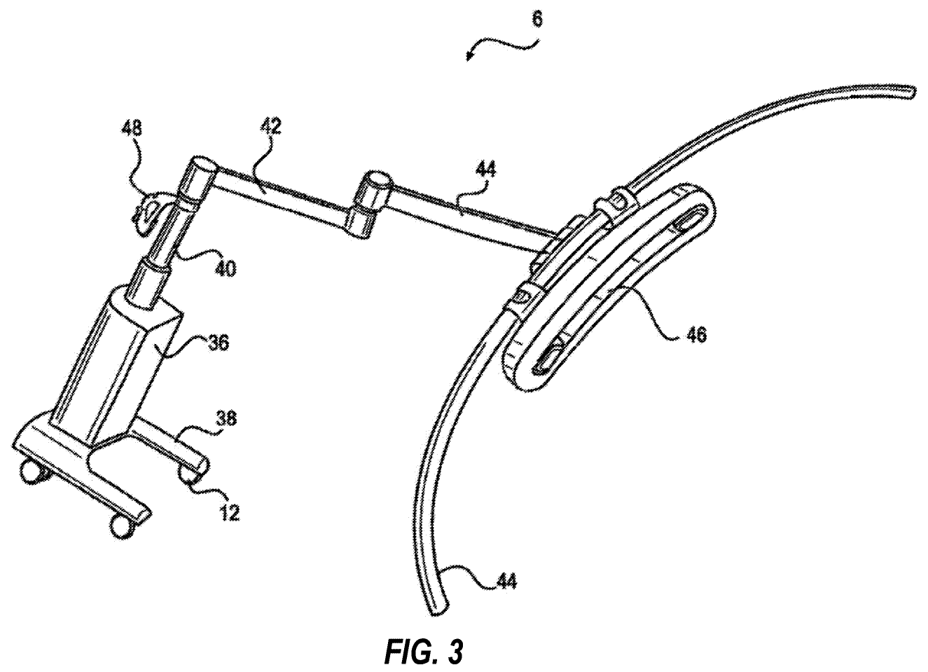

Curved rail 44 may be disposed at any suitable location on lower camera support arm 42. As illustrated in FIG. 3, curved rail 44 may attach to lower camera support arm 42 by any suitable mechanism. Suitable mechanism may be, but are not limited to nuts and bolts, ball and socket fitting, press fitting, weld, adhesion, screws, rivets, clamps, latches, and/or any combination thereof. Curved rail 44 may be of any suitable shape, a suitable shape may be a crescent, circular, oval, elliptical, and/or any combination thereof. In embodiments, curved rail 44 may be any appropriate length. An appropriate length may be about one foot to about six feet. Camera 46 may be moveably disposed along curved rail 44. Camera 46 may attach to curved rail 44 by any suitable mechanism. Suitable mechanism may be, but are not limited to rollers, brackets, braces, motors, and/or any combination thereof. Motors and rollers, not illustrated, may be used to move camera 46 along curved rail 44. As illustrated in FIG. 3, during a medical procedure, if an object prevents camera 46 from viewing one or more DRAs 52, the motors may move camera 46 along curved rail 44 using rollers. This motorized movement may allow camera 46 to move to a new position that is no longer obstructed by the object without moving camera tracking system 6. While camera 46 is obstructed from viewing DRAs 52, camera tracking system 6 may send a stop signal to surgical robot 4, display 34, and/or a tablet. The stop signal may prevent SCARA 24 from moving until camera 46 has reacquired DRAs 52. This stoppage may prevent SCARA 24 and/or end effector coupler 22 from moving and/or using medical tools without being tracked by surgical system 2.

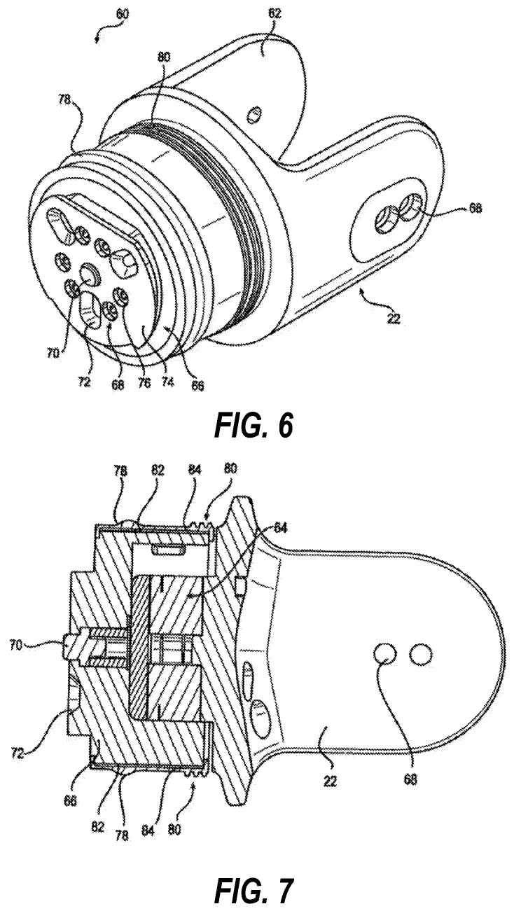

End effector coupler 22, as illustrated in FIG. 6, is configured to connect various types of passive end effectors to surgical robot 4. End effector coupler 22 can include a saddle joint 62, an activation assembly 60, a load cell 64 (FIG. 7), and a connector 66. Saddle joint 62 may attach end effector coupler 22 to SCARA 24. Saddle joint 62 may be made of any suitable material. Suitable material may be, but is not limited to metal such as titanium, aluminum, or stainless steel, carbon fiber, fiberglass, or heavy-duty plastic. Saddle joint 62 may be made of a single piece of metal which may provide end effector with additional strength and durability. The saddle joint 62 may attach to SCARA 24 by an attachment point 68. There may be a plurality of attachment points 68 disposed about saddle joint 62. Attachment points 68 may be sunk, flush, and/or disposed upon saddle joint 62. In some examples, screws, nuts and bolts, and/or any combination thereof may pass through attachment point 68 and secure saddle joint 62 to SCARA 24. The nuts and bolts may connect saddle joint 62 to a motor, not illustrated, within SCARA 24. The motor may move saddle joint 62 in any direction. The motor may further prevent saddle joint 62 from moving from accidental bumps and/or accidental touches by actively servoing at the current location or passively by applying spring actuated brakes.

The end effector coupler 22 can include a load cell 64 interposed between the saddle join 62 and a connected passive end effector. Load cell 64, as illustrated in FIG. 7 may attach to saddle joint 62 by any suitable mechanism. Suitable mechanism may be, but is not limited to, screws, nuts and bolts, threading, press fitting, and/or any combination thereof.

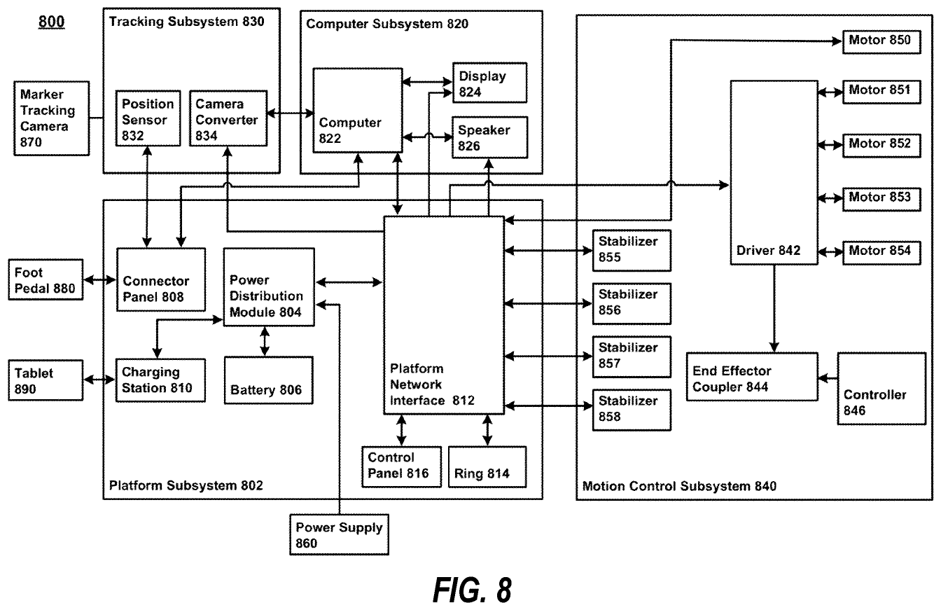

FIG. 8 illustrates a block diagram of components of a surgical system 800 according to some embodiments of the present disclosure. Referring to FIGS. 7 and 8, load cell 64 may be any suitable instrument used to detect and measure forces. In some examples, load cell 64 may be a six axis load cell, a three-axis load cell or a uniaxial load cell. Load cell 64 may be used to track the force applied to end effector coupler 22. In some embodiments the load cell 64 may communicate with a plurality of motors 850, 851, 852, 853, and/or 854. As load cell 64 senses force, information as to the amount of force applied may be distributed from a switch array and/or a plurality of switch arrays to a controller 846. Controller 846 may take the force information from load cell 64 and process it with a switch algorithm. The switch algorithm is used by the controller 846 to control a motor driver 842. The motor driver 842 controls operation of one or more of the motors. Motor driver 842 may direct a specific motor to produce, for example, an equal amount of force measured by load cell 64 through the motor. In some embodiments, the force produced may come from a plurality of motors, e.g., 850-854, as directed by controller 846. Additionally, motor driver 842 may receive input from controller 846. Controller 846 may receive information from load cell 64 as to the direction of force sensed by load cell 64. Controller 846 may process this information using a motion controller algorithm. The algorithm may be used to provide information to specific motor drivers 842. To replicate the direction of force, controller 846 may activate and/or deactivate certain motor drivers 842. Controller 846 may control one or more motors, e.g. one or more of 850-854, to induce motion of passive end effector 1100 in the direction of force sensed by load cell 64. This force-controlled motion may allow an operator to move SCARA 24 and passive end effector 1100 effortlessly and/or with very little resistance. Movement of passive end effector 1100 can be performed to position passive end effector 1100 in any suitable pose (i.e., location and angular orientation relative to defined three-dimensional (3D) orthogonal reference axes) for use by medical personnel.

Connector 66 is configured to be connectable to the base of the passive end effector 1100 and is connected to load cell 64. Connector 66 can include attachment points 68, a sensory button 70, tool guides 72, and/or tool connections/attachment points 74. Best illustrated in FIGS. 6 and 8, there may be a plurality of attachment points 68. Attachment points 68 may connect connector 66 to load cell 64. Attachment points 68 may be sunk, flush, and/or disposed upon connector 66. Attachment points 68 and 76 can be used to attach connector 66 to load cell 64 and/or to passive end effector 1100. In some examples, Attachment points 68 and 76 may include screws, nuts and bolts, press fittings, magnetic attachments, and/or any combination thereof

As illustrated in FIG. 6, a sensory button 70 may be disposed about center of connector 66. Sensory button 70 may be depressed when a passive end effector 1100 is connected to SCARA 24. Depression of sensory button 70 may alert surgical robot 4, and in turn medical personnel, that a passive end effector 1100 has been attached to SCARA 24. As illustrated in FIG. 6, guides 72 may be used to facilitate proper attachment of passive end effector 1100 to SCARA 24. Guides 72 may be sunk, flush, and/or disposed upon connector 66. In some examples there may be a plurality of guides 72 and may have any suitable patterns and may be oriented in any suitable direction. Guides 72 may be any suitable shape to facilitate attachment of passive end effector 1100 to SCARA 24. A suitable shape may be, but is not limited to, circular, oval, square, polyhedral, and/or any combination thereof. Additionally, guides 72 may be cut with a bevel, straight, and/or any combination thereof.

Connector 66 may have attachment points 74. As illustrated in FIG. 6, attachment points 74 may form a ledge and/or a plurality of ledges. Attachment points 74 may provide connector 66 a surface upon which passive end effector 1100 may clamp. In some embodiments, attachment points 74 are disposed about any surface of connector 66 and oriented in any suitable manner in relation to connector 66.

Activation assembly 60, best illustrated in FIGS. 6 and 7, may encircle connector 66. In some embodiments, activation assembly 60 may take the form of a bracelet that wraps around connector 66. In some embodiments, activation assembly 60, may be located in any suitable area within surgical system 2. In some examples, activation assembly 60 may be located on any part of SCARA 24, any part of end effector coupler 22, may be worn by medical personnel (and communicate wirelessly), and/or any combination thereof. Activation assembly 60 may be made of any suitable material. Suitable material may be, but is not limited to neoprene, plastic, rubber, gel, carbon fiber, fabric, and/or any combination thereof. Activation assembly 60 may comprise of a primary button 78 and a secondary button 80. Primary button 78 and secondary button 80 may encircle the entirety of connector 66.

Primary button 78 may be a single ridge, as illustrated in FIG. 6, which may encircle connector 66. In some examples, primary button 78 may be disposed upon activation assembly 60 along the end farthest away from saddle joint 62. Primary button 78 may be disposed upon primary activation switch 82, best illustrated on FIG. 7. Primary activation switch 82 may be disposed between connector 66 and activation assembly 60. In some examples, there may be a plurality of primary activation switches 82, which may be disposed adjacent and beneath primary button 78 along the entire length of primary button 78. Depressing primary button 78 upon primary activation switch 82 may allow an operator to move SCARA 24 and end effector coupler 22. As discussed above, once set in place, SCARA 24 and end effector coupler 22 may not move until an operator programs surgical robot 4 to move SCARA 24 and end effector coupler 22, or is moved using primary button 78 and primary activation switch 82. In some examples, it may require the depression of at least two non-adjacent primary activation switches 82 before SCARA 24 and end effector coupler 22 will respond to operator commands. Depression of at least two primary activation switches 82 may prevent the accidental movement of SCARA 24 and end effector coupler 22 during a medical procedure.

Activated by primary button 78 and primary activation switch 82, load cell 64 may measure the force magnitude and/or direction exerted upon end effector coupler 22 by an operator, i.e. medical personnel. This information may be transferred to motors within SCARA 24 that may be used to move SCARA 24 and end effector coupler 22. Information as to the magnitude and direction of force measured by load cell 64 may cause the motors to move SCARA 24 and end effector coupler 22 in the same direction as sensed by load cell 64. This force-controlled movement may allow the operator to move SCARA 24 and end effector coupler 22 easily and without large amounts of exertion due to the motors moving SCARA 24 and end effector coupler 22 at the same time the operator is moving SCARA 24 and end effector coupler 22.

Secondary button 80, as illustrated in FIG. 6, may be disposed upon the end of activation assembly 60 closest to saddle joint 62. In some examples secondary button 80 may comprise a plurality of ridges. The plurality of ridges may be disposed adjacent to each other and may encircle connector 66. Additionally, secondary button 80 may be disposed upon secondary activation switch 84. Secondary activation switch 84, as illustrated in FIG. 7, may be disposed between secondary button 80 and connector 66. In some examples, secondary button 80 may be used by an operator as a “selection” device. During a medical operation, surgical robot 4 may notify medical personnel to certain conditions by display 34 and/or light indicator 28. Medical personnel may be prompted by surgical robot 4 to select a function, mode, and/or assess the condition of surgical system 2. Depressing secondary button 80 upon secondary activation switch 84 a single time may activate certain functions, modes, and/or acknowledge information communicated to medical personnel through display 34 and/or light indicator 28. Additionally, depressing secondary button 80 upon secondary activation switch 84 multiple times in rapid succession may activate additional functions, modes, and/or select information communicated to medical personnel through display 34 and/or light indicator 28. In some examples, at least two non-adjacent secondary activation switches 84 may be depressed before secondary button 80 may function properly. This requirement may prevent unintended use of secondary button 80 from accidental bumping by medical personnel upon activation assembly 60. Primary button 78 and secondary button 80 may use software architecture 86 to communicate commands of medical personnel to surgical system 2.

FIG. 8 illustrates a block diagram of components of a surgical system 800 configured according to some embodiments of the present disclosure, and which may correspond to the surgical system 2 above. Surgical system 800 includes platform subsystem 802, computer subsystem 820, motion control subsystem 840, and tracking subsystem 830. Platform subsystem 802 includes battery 806, power distribution module 804, connector panel 808, and charging station 810. Computer subsystem 820 includes computer 822, display 824, and speaker 826. Motion control subsystem 840 includes driver circuit 842, motors 850, 851, 852, 853, 854, stabilizers 855, 856, 857, 858, end effector connector 844, and controller 846. Tracking subsystem 830 includes position sensor 832 and camera converter 834. Surgical system 800 may also include a removable foot pedal 880 and removable tablet computer 890.

Input power is supplied to surgical system 800 via a power source which may be provided to power distribution module 804. Power distribution module 804 receives input power and is configured to generate different power supply voltages that are provided to other modules, components, and subsystems of surgical system 800. Power distribution module 804 may be configured to provide different voltage supplies to connector panel 808, which may be provided to other components such as computer 822, display 824, speaker 826, driver 842 to, for example, power motors 850-854 and end effector coupler 844, and provided to camera converter 834 and other components for surgical system 800. Power distribution module 804 may also be connected to battery 806, which serves as temporary power source in the event that power distribution module 804 does not receive power from an input power. At other times, power distribution module 804 may serve to charge battery 806.

Connector panel 808 may serve to connect different devices and components to surgical system 800 and/or associated components and modules. Connector panel 808 may contain one or more ports that receive lines or connections from different components. For example, connector panel 808 may have a ground terminal port that may ground surgical system 800 to other equipment, a port to connect foot pedal 880, a port to connect to tracking subsystem 830, which may include position sensor 832, camera converter 834, and marker tracking cameras 870. Connector panel 808 may also include other ports to allow USB, Ethernet, HDMI communications to other components, such as computer 822.

Control panel 816 may provide various buttons or indicators that control operation of surgical system 800 and/or provide information from surgical system 800 for observation by an operator. For example, control panel 816 may include buttons to power on or off surgical system 800, lift or lower vertical column of support arm 16, and lift or lower stabilizers 855-858 that may be designed to engage casters (e.g., powered wheels 12) to lock surgical system 800 from physically moving. Other buttons may stop surgical system 800 in the event of an emergency, which may remove all motor power and apply mechanical brakes to stop all motion from occurring. Control panel 816 may also have indicators notifying the operator of certain system conditions such as a line power indicator or status of charge for battery 806.

Computer 822 of computer subsystem 820 includes an operating system and software to operate assigned functions of surgical system 800. Computer 822 may receive and process information from other components (for example, tracking subsystem 830, platform subsystem 802, and/or motion control subsystem 840) in order to display information to the operator. Further, computer subsystem 820 may provide output through the speaker 826 for the operator. The speaker may be part of the surgical robot, part of a head-mounted display component, or within another component of the surgical system 2. The display 824 may correspond to the display 34 shown in FIGS. 1 and 2, or may be a head-mounted display which projects images onto a see-through display screen which forms an augmented reality (AR) image that is overlaid on real-world objects viewable through the see-through display screen.

Tracking subsystem 830 may include position sensor 832 and camera converter 834. Tracking subsystem 830 may correspond to the camera tracking system 6 of FIG. 3. The marker tracking cameras 870 operate with the position sensor 832 to determine the pose of DRAs 52. This tracking may be conducted in a manner consistent with the present disclosure including the use of infrared or visible light technology that tracks the location of active or passive elements of DRAs 52, such as LEDs or reflective markers, respectively. The location, orientation, and position of structures having these types of markers, such as DRAs 52, is provided to computer 822 and which may be shown to an operator on display 824. For example, as shown in FIGS. 4 and 5, a surgical saw 1240 having a DRA 52 or which is connected to an end effector coupler 22 having a DRA 52 tracked in this manner (which may be referred to as a navigational space) may be shown to an operator in relation to a three dimensional image of a patient's anatomical structure.

Motion control subsystem 840 may be configured to physically move vertical column 16, upper arm 18, lower arm 20, or rotate end effector coupler 22. The physical movement may be conducted through the use of one or more motors 850-854. For example, motor 850 may be configured to vertically lift or lower vertical column 16. Motor 851 may be configured to laterally move upper arm 18 around a point of engagement with vertical column as shown in FIG. 2. Motor 852 may be configured to laterally move lower arm 20 around a point of engagement with upper arm 18 as shown in FIG. 2. Motors 853 and 854 may be configured to move end effector coupler 22 to provide translational movement and rotation along in about three-dimensional axes. The surgical planning computer 910 shown in FIG. 9 can provide control input to the controller 846 that guides movement of the end effector coupler 22 to position a passive end effector, which is connected thereto, with a planned pose (i.e., location and angular orientation relative to defined 3D orthogonal reference axes) relative to an anatomical structure that is to be cut during a surgical procedure. Motion control subsystem 840 may be configured to measure position of the passive end effector structure using integrated position sensors (e.g. encoders). In one of the embodiments, position sensors are directly connected to at least one joint of the passive end effector structure, but may also be positioned in another location in the structure and remotely measure the joint position by interconnection of a timing belt, a wire, or any other synchronous transmission interconnection.

FIG. 9 illustrates a block diagram of a surgical system computer platform 900 that includes a surgical planning computer 910 which may be separate from and operationally connected to a surgical robot 800 or at least partially incorporated therein according to some embodiments of the present disclosure. Alternatively, at least a portion of operations disclosed herein for the surgical planning computer 910 may be performed by components of the surgical robot 800 such as by the computer subsystem 820.

Referring to FIG. 9, the surgical planning computer 910 includes a display 912, at least one processor circuit 914 (also referred to as a processor for brevity), at least one memory circuit 916 (also referred to as a memory for brevity) containing computer readable program code 918, and at least one network interface 920 (also referred to as a network interface for brevity). The network interface 920 can be configured to connect to a C-Arm imaging device 104 in FIG. 10, an O-Arm imaging device 106 in FIG. 11, another medical imaging device, an image database 950 of medical images, components of the surgical robot 800, and/or other electronic equipment.

When the surgical planning computer 910 is at least partially integrated within the surgical robot 800, the display 912 may correspond to the display 34 of FIG. 2 and/or the tablet 890 of FIG. 8 and/or a head-mounted display, the network interface 920 may correspond to the platform network interface 812 of FIG. 8, and the processor 914 may correspond to the computer 822 of FIG. 8.

The processor 914 may include one or more data processing circuits, such as a general purpose and/or special purpose processor, e.g., microprocessor and/or digital signal processor. The processor 914 is configured to execute the computer readable program code 918 in the memory 916 to perform operations, which may include some or all of the operations described herein as being performed by a surgical planning computer.

The processor 914 can operate to display on the display device 912 an image of a bone that is received from one of the imaging devices 104 and 106 and/or from the image database 950 through the network interface 920. The processor 914 receives an operator's definition of where an anatomical structure, i.e. one or more bones, shown in one or more images is to be cut, such as by an operator touch selecting locations on the display 912 for planned surgical cuts or using a mouse-based cursor to define locations for planned surgical cuts.

The surgical planning computer 910 enables anatomy measurement, useful for knee surgery, like measurement of various angles determining center of hip, center of angles, natural landmarks (e.g. transepicondylar line, Whitesides line, posterior condylar line etc.), etc. Some measurements can be automatic while some others involve human input or assistance. This surgical planning computer 910 allows an operator to choose the correct implant for a patient, including choice of size and alignment. The surgical planning computer 910 enables automatic or semi-automatic (involving human input) segmentation (image processing) for CT images or other medical images. The surgical plan for a patient may be stored in a cloud-based server for retrieval by the surgical robot 800. During the surgery, the surgeon will choose which cut to make (e.g. posterior femur, proximal tibia etc.) using a computer screen (e.g. touchscreen) or augmented reality interaction via, e.g., a head-mounted display. The surgical robot 4 may automatically move the surgical saw blade to a planned position so that a target plane of planned cut is optimally placed within a workspace of the passive end effector interconnecting the surgical saw blade and the robot arm 20. Command enabling movement can be given by user using various modalities, e.g. foot pedal.

In some embodiments, the surgical system computer platform 900 can use two DRAs to tracking patient anatomy position: one on patient tibia and one on patient femur. The platform 900 may use standard navigated instruments for the registration and checks (e.g., a pointer similar to the one used in Globus ExcelsiusGPS system for spine surgery). Tracking markers allowing for detection of DRAs movement in reference to tracked anatomy can be used as well.

An important difficulty in knee surgery is how to plan the position of the implant in the knee and many surgeons struggle with to do it on a computer screen which is a 2D representation of 3D anatomy. The platform 900 could address this problem by using an augmented reality (AR) head-mounted display to generate an implant overlay around the actual patient knee. For example, the surgeon can be operationally displayed a virtual handle to grab and move the implant to a desired pose and adjust planned implant placement. Afterward, during surgery, the platform 900 could render the navigation through the AR head-mounted display to show surgeon what is not directly visible. Also, the progress of bone removal, e.g., depth or cut, can be displayed in real-time. Other features that may be displayed through AR can include, without limitation, gap or ligament balance along a range of joint motion, contact line on the implant along the range of joint motion, ligament tension and/or laxity through color or other graphical overlays, etc.

The surgical planning computer 910, in some embodiments, can allow planning for use of standard implants, e.g., posterior stabilized implants and cruciate retaining implants, cemented and cementless implants, revision systems for surgeries related to, for example, total or partial knee and/or hip replacement and/or trauma.

The processor 914 may graphically illustrate on the display 912 one or more cutting planes intersecting the displayed anatomical structure at the locations selected by the operator for cutting the anatomical structure. The processor 914 also determines one or more sets of angular orientations and locations where the end effector coupler 22 must be positioned so a cutting plane of the surgical saw blade will be aligned with a target plane to perform the operator defined cuts, and stores the sets of angular orientations and locations as data in a surgical plan data structure. The processor 914 uses the known range of movement of the tool attachment mechanism of the passive end effector to determine where the end effector coupler 22 attached to the robot arm 20 needs to be positioned.

The computer subsystem 820 of the surgical robot 800 receives data from the surgical plan data structure and receives information from the camera tracking system 6 indicating a present pose of an anatomical structure that is to be cut and indicating a present pose of the passive end effector and/or surgical saw tracked through DRAs. The computer subsystem 820 determines a pose of the target plane based on the surgical plan defining where the anatomical structure is to be cut and based on the pose of the anatomical structure. The computer subsystem 820 generates steering information based on comparison of the pose of the target plane and the pose of the surgical saw. The steering information indicates where the passive end effector needs to be moved so the cutting plane of the saw blade becomes aligned with the target plane and the saw blade becomes positioned a distance from the anatomical structure to be cut that is within the range of movement of the tool attachment mechanism of the passive end effector.

As explained above, a surgical robot includes a robot base, a robot arm connected to the robot base, and at least one motor operatively connected to move the robot arm relative to the robot base. The surgical robot also includes at least one controller, e.g. the computer subsystem 820 and the motion control subsystem 840, connected to the at least one motor and configured to perform operations.

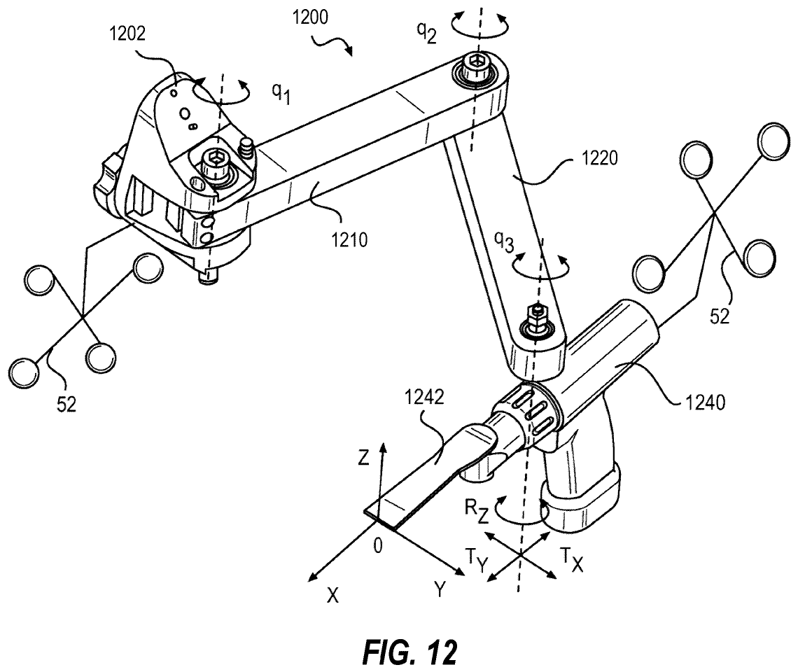

As will be explained in further detail below with regard to FIGS. 12-19, a passive end effector includes a base configured to attach to an activation assembly of the robot arm, a first mechanism, and a second mechanism. The first mechanism extends between a rotatable connection to the base and a rotatable connection to a tool attachment mechanism. The second mechanism extends between a rotatable connection to the base and a rotatable connection to the tool attachment mechanism. The first and second mechanisms pivot about the rotatable connections which may be configured to constrain movement of the tool attachment mechanism to a range of movement within a working plane. The rotatable connections may be pivot joints allowing 1 degree-of-freedom (DOF) motion, universal joints allowing 2 DOF motions, or ball joints allowing 3 DOF motions. The tool attachment mechanism is configured to connect to the surgical saw comprising a saw blade for cutting. The first and second mechanisms may be configured to constrain a cutting plane of the saw blade to be parallel to the working plane.

In some embodiments, the operations performed by the at least one controller of the surgical robot also includes controlling movement of the at least one motor based on the steering information to reposition the passive end effector so the cutting plane of the saw blade becomes aligned with the target plane and the saw blade becomes positioned the distance from the anatomical structure to be cut that is within the range of movement of the tool attachment mechanism of the passive end effector. The steering information may be displayed to guide an operator's movement of the surgical saw and/or may be used by the at least one controller to automatically move the surgical saw.

In one embodiment, the operations performed by the at least one controller of the surgical robot also includes providing the steering information to a display device for display to guide operator movement of the passive end effector so the cutting plane of the saw blade becomes aligned with the target plane and so the saw blade becomes positioned the distance from the anatomical structure, which is to be cut, that is within the range of movement of the tool attachment mechanism of the passive end effector. The display device may correspond to the display 824 (FIG. 8), the display 34 of FIG. 1, and/or a head-mounted display.

For example, the steering information may be displayed on a head-mounted display which projects images onto a see-through display screen which forms an augmented reality image that is overlaid on real-world objects viewable through the see-through display screen. The operations may display a graphical representation of the target plane with a pose overlaid on a bone and with a relative orientation there between corresponding to the surgical plan for how the bone is planned to be cut. The operations may alternatively or additionally display a graphical representation of the cutting plane of the saw blade so that an operator may more easily align the cutting plane with the planned target plane for cutting the bone. The operator may thereby visually observe and perform movements to align the cutting plane of the saw blade with the target plane so the saw blade becomes positioned at the planned pose relative to the bone and within a range of movement of the tool attachment mechanism of the passive end effector.

An automated imaging system can be used in conjunction with the surgical planning computer 910 and/or the surgical system 2 to acquire pre-operative, intra-operative, post-operative, and/or real-time image data of a patient. Example automated imaging systems are illustrated in FIGS. 10 and 11. In some embodiments, the automated imaging system is a C-arm 104 (FIG. 10) imaging device or an O-Arm® 106 (FIG. 11). (O-Arm® is copyrighted by Medtronic Navigation, Inc. having a place of business in Louisville, Colo., USA) It may be desirable to take x-rays of a patient from a number of different positions, without the need for frequent manual repositioning of the patient which may be required in an x-ray system. C-arm 104 x-ray diagnostic equipment may solve the problems of frequent manual repositioning and may be well known in the medical art of surgical and other interventional procedures. As illustrated in FIG. 10, a C-arm includes an elongated C-shaped member terminating in opposing distal ends 112 of the “C” shape. C-shaped member is attached to an x-ray source 114 and an image receptor 116. The space within C-arm 104 of the arm provides room for the physician to attend to the patient substantially free of interference from the x-ray support structure.

The C-arm is mounted to enable rotational movement of the arm in two degrees of freedom, (i.e. about two perpendicular axes in a spherical motion). C-arm is slidably mounted to an x-ray support structure, which allows orbiting rotational movement of the C-arm about its center of curvature, which may permit selective orientation of x-ray source 114 and image receptor 116 vertically and/or horizontally. The C-arm may also be laterally rotatable, (i.e. in a perpendicular direction relative to the orbiting direction to enable selectively adjustable positioning of x-ray source 114 and image receptor 116 relative to both the width and length of the patient). Spherically rotational aspects of the C-arm apparatus allow physicians to take x-rays of the patient at an optimal angle as determined with respect to the particular anatomical condition being imaged.

The O-Arm® 106 illustrated in FIG. 11 includes a gantry housing 124 which may enclose an image capturing portion, not illustrated. The image capturing portion includes an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of the patient to be acquired from multiple directions or in multiple planes.

The O-Arm® 106 with the gantry housing 124 has a central opening for positioning around an object to be imaged, a source of radiation that is rotatable around the interior of gantry housing 124, which may be adapted to project radiation from a plurality of different projection angles. A detector system is adapted to detect the radiation at each projection angle to acquire object images from multiple projection planes in a quasi-simultaneous manner. The gantry may be attached to a support structure O-Arm® support structure, such as a wheeled mobile cart with wheels, in a cantilevered fashion. A positioning unit translates and/or tilts the gantry to a planned position and orientation, preferably under control of a computerized motion control system. The gantry may include a source and detector disposed opposite one another on the gantry. The source and detector may be secured to a motorized rotor, which may rotate the source and detector around the interior of the gantry in coordination with one another. The source may be pulsed at multiple positions and orientations over a partial and/or full three hundred and sixty degree rotation for multi-planar imaging of a targeted object located inside the gantry. The gantry may further comprise a rail and bearing system for guiding the rotor as it rotates, which may carry the source and detector. Both and/or either O-Arm® 106 and C-arm 104 may be used as automated imaging system to scan a patient and send information to the surgical system 2.

Images captured by the automated imaging system can be displayed a display device of the surgical planning computer 910, the surgical robot 800, and/or another component of the surgical system 2.

Various embodiments of passive end effectors that are configured for use with a surgical system are now described in the context of FIGS. 12-21.

As will be explained in further detail below, the various passive end effectors illustrated in FIGS. 12-18E and 21 may each include a base, a first planer mechanism, and a second planer mechanism. The base is configured to attach to an end effector coupler (e.g., end effector coupler 22 in FIGS. 4 and 5) of a robot arm (e.g., robot arm 20 in FIGS. 1 and 2) positioned by a surgical robot. Various clamping mechanisms may be used to firmly attach the base to the end effector coupler, removing backlash and ensuring suitable stiffness. Irreversible clamping mechanisms which may be used to attach the base to the end effector coupler can include but are not limited to toggle joint mechanisms or irreversible locking screw(s). A user may use an additional tool, such as but not limited to, a screwdriver, torque wrench, or driver to activate or tighten the clamping mechanism. The first mechanism extends between a rotatable connection to the base of a two and a rotatable connection to a tool attachment mechanism. The second mechanism extends between a rotatable connection to the base and a rotatable connection to the tool attachment mechanism. The first and second mechanisms pivot about the rotatable connections. The rotatable connections may be pivot joints allowing 1 degree-of-freedom (DOF) motion, universal joints allowing 2 DOF motions, or ball joints allowing 3 DOF motions. When pivot joints are used the first and second mechanisms can be configured to constrain movement of the tool attachment mechanism to a range of movement within a working plane. The tool attachment mechanism is configured to connect to a surgical saw having a saw blade that is configured to oscillate for cutting. The first and second mechanisms may be configured, e.g., via pivot joints having 1 DOF motion, to constrain a cutting plane of the saw blade to be parallel to the working plane. The tool attachment mechanism may connect to the surgical saw or saw blade through various mechanisms that can include, but are not limited to, a screw, nut and bolt, clamp, latch, tie, press fit, or magnet. A DRA can be connected to the tool attachment mechanism or the surgical saw to enable tracking of a pose of the saw blade by the camera tracking system 6 (FIG. 3).

As explained above, a surgical system (e.g., surgical system 2 in FIGS. 1 and 2) includes a surgical robot (e.g., surgical robot 4 in FIGS. 1 and 2) and a tracking system (e.g., camera tracking system 6 in FIGS. 1 and 3) that is configured to determine a pose of an anatomical structure that is to be cut by the saw blade and to determine a pose of the saw blade. The surgical robot includes a robot base, a robot arm that is rotatably connected to the robot base and configured to position the passive end effector. At least one motor is operatively connected to move the robot arm relative to the robot base. At least one controller is connected to the at least one motor and configured to perform operations that include determining a pose of a target plane based on a surgical plan defining where the anatomical structure is to be cut and based on the pose of the anatomical structure, where the surgical plan may be generated by the surgical planning computer 910 of FIG. 9 based on input from an operator, e.g., surgeon or other surgery personnel. The operations further include generating steering information based on comparison of the pose of the target plane and the pose of the surgical saw. The steering information indicates where the passive end effector needs to be moved to position the working plane of the passive end effector so the cutting plane of the saw blade is aligned with the target plane.

In some further embodiments, the operations performed by the at least one controller further include controlling movement of the at least one motor based on the steering information to reposition the passive end effector so the cutting plane of the saw blade becomes aligned with the target plane and the saw blade becomes positioned a distance from the anatomical structure to be cut that is within the range of movement of the tool attachment mechanism of the passive end effector.

The operations may include providing the steering information to a display device for display to guide operator movement of the passive end effector so the cutting plane of the saw blade becomes aligned with the target plane and so the saw blade becomes positioned a distance from the anatomical structure, which is to be cut, that is within the range of movement of the tool attachment mechanism of the passive end effector.

As explained above, some surgical systems can include head-mounted display devices that can be worn by a surgeon, nurse practitioner, and/or other persons assisting with the surgical procedure. The surgical systems can display information that allows the wearer to position the passive end effector more accurately and/or to confirm that it has been positioned accurately with the saw blade aligned with the target plane for cutting a planned location on an anatomical structure. The operation to provide the steering information to the display device, may include configuring the steering information for display on a head-mounted display device having a see-through display screen that displays the steering information as an overlay on the anatomical structure that is to be cut to guide operator movement of the passive end effector so the cutting plane of the saw blade becomes aligned with the target plane and the saw blade becomes positioned the distance from the anatomical structure within the range of movement of the tool attachment mechanism of the passive end effector.

The operation to configure the steering information for display on the head-mounted display device, may include generating a graphical representation of the target plane that is displayed as an overlay anchored to and aligned with the anatomical structure that is to be cut, and generating another graphical representation of the cutting plane of the saw blade that is displayed as an overlay anchored to and aligned with the saw blade. A wearer may thereby move the surgical saw to provide visually observed alignment between the graphically rendered target plane and the graphically rendered cutting plane.

The operation to configure the steering information for display on the head-mounted display device, may include generating a graphical representation a depth of cut made by the saw blade into a graphical representation of the anatomical structure being cut. Thus, the wearer can use the graphical representation of depth of cut to better monitor how the saw blade is cutting through bone despite direct observation of the cutting being obstructed by tissue or other structure.

The tracking system can be configured to determine the pose of the anatomical structure that is to be cut by the saw blade based on determining a pose of tracking markers, e.g., DRAs, that are attached to the anatomical structure, and can be configured to determine a pose of the surgical saw based on determining a pose of tracking markers connected to at least one of the surgical saw and the passive end effector. The tracking system can be configured to determine the pose of the surgical saw based on rotary position sensors which are configured to measure rotational positions of the first and second mechanisms during movement of the tool attachment mechanism within the working plane. As explained above, position sensors may be directly connected to at least one joint of the passive end effector structure, but may also be positioned in another location in the structure and remotely measure the joint position by interconnection of a timing belt, a wire, or any other synchronous transmission interconnection. Additionally the pose of the saw blade can be determined based on the tracking markers attached to the structure base, position sensors in the passive structure and kinematic model of the structure.

The various passive end effectors disclosed herein can be sterilizable or non-sterile (covered by a sterile drape) passive 3DOF (Degree Of Freedom) mechanical structures allowing mechanical guidance of a surgical saw or saw blade, such as a sagittal saw, along two translations in a plane parallel to the saw blade (defining the cut plane), and one rotation perpendicular to this cut plane (instrument orientation). During the surgery, the surgical robot 4 moves the end effector coupler 22, and the passive end effector and surgical saw attached there, automatically to a position close to a knee or other anatomical structure, so that all bone to be cut is within the workspace of the passive end effector. This position depends on the cut to be made and the surgery planning and implant construction. The passive end effector can have 3 DOF to guide sagittal saw or saw blade on the cutting plane providing two translation (X and Y directions) and a rotation (around Z axis) as shown in FIG. 12.

When the surgical robot 4 achieves a planned position, it holds the position (either on brakes or active motor control) and does not move during the particular bone cut. It is the passive end effector that allows movement of the saw blade of the surgical saw along the planned target plane. Such planar cuts are particularly useful for classical total knee arthroplasty where all bone cuts are planar. In partial knee arthroplasty there are special types of implants, called “on-lay” which can be used in conjunction with saw-prepared bone surfaces. The various passive end effectors have mechanical structure that can ensure precision of guidance during cuts, with higher precision than classical jigs, and provide sufficient range of workspace range to cut all the bone that is planned and while provide sufficient transverse stiffness (corresponding to locked DOF) despite possibly significant amount of vibrations originating from the surgical saw in addition to forces applied by the surgeon and bone reactionary forces.

As the same time, it is preferable to measure the passive end effector position because it enables the surgical robot 4 to inform the surgeon how much bone has been removed (procedure advancement). One way to provide real-time information on bone removal is for the surgical robot 4 to measure where the saw blade passed in reference to the bone because the blade can pass only where the bone has been cut. To measure saw blade position a DRA can be mounted to the surgical saw and/or the passive end effector. This enables direct or indirect measurement of the saw position in 3D space. An alternative way to measure saw blade position is to integrate position (rotation or translation) sensors (e.g. encoders, resolvers) into position information of the passive end effector in order to calculate position of the saw blade using a mathematical model of a defined relationship between location of the passive end effector geometry and the tip of the saw blade.

In one embodiment, a conventional sagittal saw mechanism can be used with the surgical system computer platform 900 with little or no changes. The potential changes would involve adapting an external shield to enable easy attachment of the surgical saw to the passive end effector but would not necessarily involve changes in the internal mechanics. The passive end effector may be configured to connect to a conventional sagittal saw provided by, for example, DeSoutter company. In addition, the saw blade may be directly attached to the passive end effector without the saw handpiece.

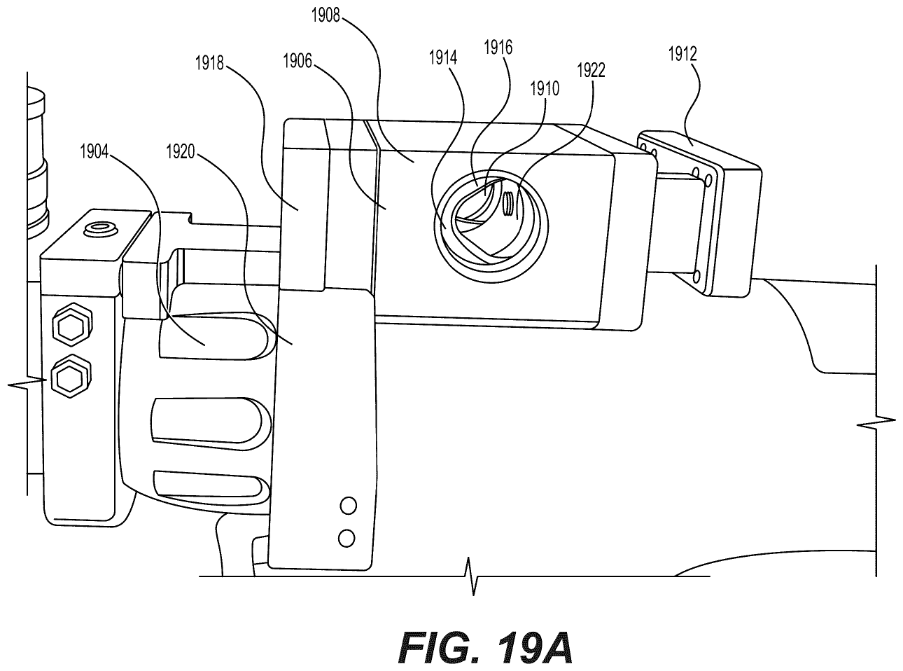

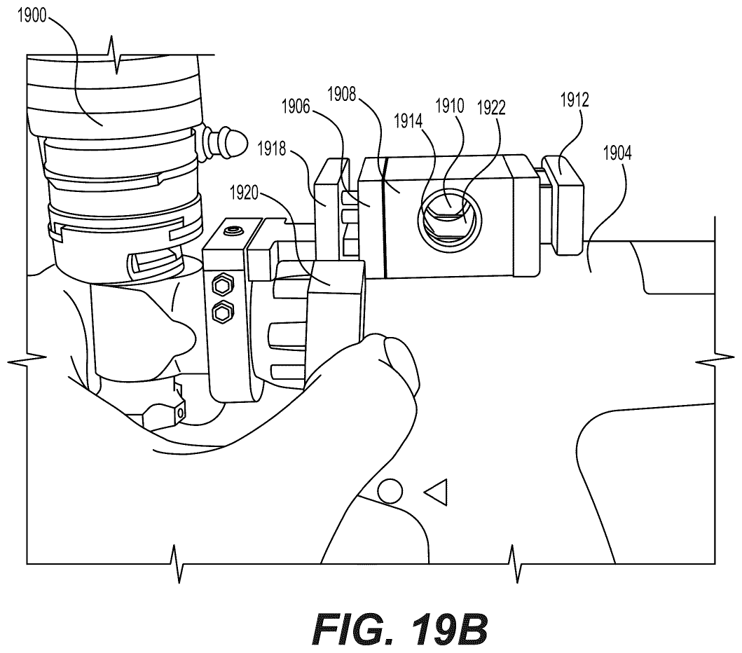

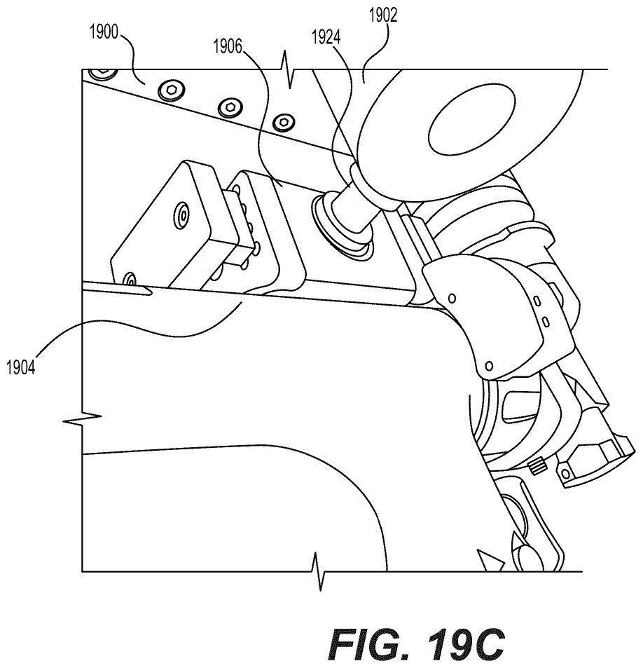

To prevent the saw from unintentional passive end effector movement when the surgical robot 4 positions the passive end effector, e.g., to prevent the surgical saw from falling on the patient due to gravitational forces, the passive end effector can include a lock mechanism that moves between engaged and disengaged operations. While engaged, the lock mechanism prevents movement of the saw blade with respect to the robot end effector coupler, either directly by locking the degree of freedoms (DOFs) of the surgical saw, or indirectly by braking or locking specifics joints of the passive end effector. While disengaged, the first and second mechanisms of the passive end effector can be moved relative to the base without interference from the lock mechanism. The lock mechanism, described in greater detail below with respect to FIGS. 19A-19C, may also be used when a surgeon holds the surgical saw and controls the surgical robot 4 movement by applying forces and torques to the surgical saw. The surgical robot 4, using the load cell 64 of FIGS. 6 and 7 integrated in the distal end of the robot arm 20, measures forces and torques that are applied and generates responsive forces and torques on the robot arm 20 so the surgeon can more easily move the passive end effector back and forth, left and right, apply rotations around various axes.

An embodiment of a passive end effector is shown in FIG. 12. The passive end effector 1200 includes a base 1202 that is configured to attach to an end effector coupler (e.g., end effector coupler 22 in FIGS. 4 and 5) of a robot arm (e.g., robot arm 18 in FIGS. 1 and 2) positioned by a surgical robot. The passive end effector 1200 further includes a first link segment 1210 and a second link segment 1220. The first link segment 1210 extends between a rotatable connection to the base 1202 and a rotatable connection to one end of the second link segment 1220. Another end of the second link segment 1220 is rotatably connected to a tool attachment mechanism. The rotational axes q1, q2 and q3 are parallel to each other so as to provide a planar cutting plane for the blade 1242. Thus, the 3 DOF motion of the saw 1240 includes x-direction Tx, y-direction Ty and rotational direction about z-axis Rz. One or more of the rotatable connections disclosed for this embodiment may be pivot joints allowing 1 DOF motion, universal joints allowing 2 DOF motions, or ball joints allowing 3 DOF motions.

The tracking markers (e.g., DRAs 52) attached to the end effector base 1202 and the saw 1240 along with the tracking markers on the bones (e.g., tibia and femur) can be used to precisely and continuously monitor the real-time location of the blade 1242 and blade tip relative to the patient bone being cut. Although not explicitly shown in other figures, the tracking markers can be attached to the saw 1240 and all end effectors (e.g., end effector 1200, end effector arm 1400, etc.) in all embodiments to track the location of the blade relative to the patient bone being cut. Although not shown, alternatively or in addition to the tracking markers, encoders can be positioned in each of the link segments 1210 and 1220 to determine precisely where the saw blade tip is at all times.

### Example Surgical Procedure

An example surgical procedure using the surgical robot 4 in an Operating Room (OR) can include:

### Optional Step: Surgery is Pre-Operatively Planned Based on Medical Images

The surgical robot 4 system is outside the Operating Room (OR). The nurse brings the system to the OR when patient is being prepared for the surgery.

The nurse powers on the robot and deploys the robot arm. Nurse verifies precision of robotic and tracking systems.

In the case of a sterilized passive end effector, the scrub nurse puts a sterile drape on the robot arm and mounts the passive end effector with the sagittal saw on the robot arm. The scrub nurse locks the passive end effector with a lock mechanism. Scrub nurse attached DRAs to passive structure through the drape (if necessary). For a non-sterilized passive end effector, the drape is placed after attachment of the passive end effector on the robot arm, the DRAs are attached to the passive end effector with the drape intervening therebetween, and a sterile saw or saw blade is attached to the passive end effector with the drape intervening therebetween. The lock mechanism is engaged, in order to fix position of the saw blade with respect to the end effector coupler.

The surgeon attaches navigation markers to the patient's bone(s), e.g., tibia and femur. The bones are registered with the camera tracking system 6 using, e.g., Horn point-to-point algorithm, surface matching or other algorithms. A soft-tissue balance assessment may be performed, whereby the system allows surgeon to assess balance of soft tissue in the operating room, e.g., by tracking relative movement of femur and tibia when surgeon applies forces in different directions (e.g. varus/valgus stress). Soft-tissue balance information can be used to alter surgical plan (e.g. move implant parts, change implant type etc.).

When surgeon is ready to cut the bone, the scrub nurse brings the surgical robot 4 to the operating table close to the knee to be operated and stabilizes the surgical robot 4 on the floor. The system may operate to guide nurse in finding robot 4 position so that all cut planes are in robot and passive structure workspace.

The surgeon selects on the screen of the surgical robot 4 the different parameters according to the planning of the surgery to do the first cut (bone to be cut, cutting plan desired, etc.).

The surgical robot 4 automatically moves the robot arm 20 to reposition the passive end effector so the cutting plane of the saw blade becomes aligned with the target plane and the saw blade becomes positioned a distance from the anatomical structure to be cut that is within the range of movement of the tool attachment mechanism of the passive end effector.

The surgeon unlocks the passive end effector.

The surgeon performs the cut constrained to the cutting plane provide by the passive end effector. The surgical robot 4 may provide real-time display of the tracked location of the saw blade relative to bone so the surgeon can monitor progress of bone removal. In one way, the tracking subsystem processes in real-time the location of the saw relative to the bone based on camera images and various tracking markers attached to the saw, robot arm, end effector, femur and tibia. The surgeon can then lock the passive end effector using the lock mechanism upon completion of the cut.

The surgeon selects on the screen the next cut to be executed and proceeds as before.

The surgeon may perform a trial implant placement and intermediate soft-tissue balance assessment and based thereon may change the implant plan and associated cuts.

Following completion of all cuts, the nurse removes the surgical robot 4 from the operating table and unmounts the passive end effector from the robot arm.

The surgeon places the implants and finishes the surgery.

In step 9 above, a physician may have a difficult time to visually confirm the progress of the cut due to tissue and ligaments around the bone and debris being created from the cut, and other surgical instruments near the bone. Even if the visual confirmation may be acceptable, there are areas of the bone the physician cannot see such as the posterior portion of the bone being cut.

Advantageously, one robotic system embodiment of the present invention provides a way for the physician to confirm the progress of the bone being cut in multiple dimensions. The camera tracking system 6 along with the tracking markers attached to the end effector base 1202, robotic arm 20 and saw 1240 allows the tracking subsystem 830 and the computer subsystem 820 to calculate in real-time the precise position of the saw blade relative to bone so the surgeon can monitor progress of bone removal. In this and other embodiments, a saw 1240 is used as an example of a handheld surgical device that is attached to and guided by a passive end effector arm. It should be understood, however, that other handheld surgical devices, such as drills, scalpels, drivers, etc. may also be used with the embodiments herein, as desired.

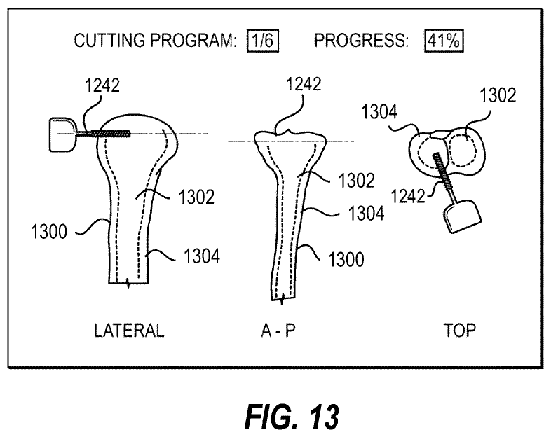

FIG. 13 is a screenshot of a display showing the progress of bone cuts during a surgical procedure. FIG. 13 shows the subsystems 830 and 820 displaying three images: lateral, A-P and top views. In each image, the real-time location of the saw blade 1242 relative to the bone (e.g., tibia 1300) is displayed on the display 34. The lateral and top views can be especially useful for the physician as they display the saw blade position which cannot be easily seen. At the top portion of the display, the computer subsystem 830, 820 displays the number of cutting programs and what program it is currently running. For example as the screenshot shows, the physician may have programmed 6 planar cuts and the current cutting program is the first one. Also, because the subsystems 830 and 820 can track with tracking markers where the blade may have travelled, it can determine how much of the bone cutting (area that was cut) for a particular cutting program has been completed and the percentage of progress is displayed in the display 34. The bone image itself is preferably derived from actual images of the patient's body for a more accurate representation. The bone image is augmented by the subsystem 820 with a contour line that shows the cortical bone 1304 and spongy bone 1302. This can be important for a physician as the amount of resistance to cutting varies greatly between the two types of bones.

If an augmented reality (AR) head-mounted display is used, the computer subsystem 820 can generate the same contour line showing the cortical and spongy bones and superimpose it over the actual leg continuously as the physician moves his/her head. The area that has been already cut can be overlaid over the actual bone in a dark shade. Moreover, the implant to be inserted over the cut area can also be overlaid on the bone to show the physician that the cutting is being done correctly along the plane of the implant. This is all possible because the subsystems 830 and 820 can track the position of the blade and history of its movement relative to the bone with the tracking markers and the camera subsystem.

As discussed with respect to the examples described above, knee surgeries may require to resect planar surfaces on the femur and the tibia, with location and orientation given by the implant that will further be placed on the resected surfaces. To perform these resections, a powered handpiece having a cutting element such as the oscillating sagittal saw blade of saw 1240 of FIG. 12, may be held by a surgeon, with a passive end effector arm, such as the passive end effector 1200 of FIG. 12, maintaining the saw within a fixed resection plane while allowing the blade three degrees of freedom within the plane, i.e., an x-direction and y-direction parallel to the resection plane, and a rotational direction about a rotation axis perpendicular to the resection plane.

In order to achieve sufficient resection accuracy, it is desirable to provide the passive end effector 1200 with a high level of transverse stiffness. For example, aluminum is one suitable material for the passive end effector 1200 due to high stiffness as well as other factors, such as manufacturing cost. The use of aluminum and/or other high-stiffness materials may impact the weight of these structural parts, however.

During knee surgery, the saw 1240 and the end effector 1200 are both positioned in the planned resection plane with respect to the patient anatomy. When the resection plane is horizontal, the gravity force due to the weight of the end effector 1200 is entirely supported by the joint bearings (e.g., of joints 1403, 1443, 1445) of the end effector 1200. However, it is common for many resection operations to employ a resection plane that is inclined with respect to the horizontal plane, up to and including 90 degrees, i.e., vertical. In such configurations, the weight of the link segments 1210, 1220 of the end effector 1200 may be partially or totally supported by the surgeon's hand. Thus, to maintain the saw 1240 in a correct position, in addition to supporting the weight of the saw 1240, the surgeon also applies a reaction force in the direction of the end effector 1200 sufficient to compensate for the vertical gravitational force on the end effector 1200. This may lead to a significant increase of perceived weight for the saw 1240, which negatively impacts usability.

The kinematic behavior of the end effector 1200 may also leads to unacceptable risks, particularly when the resection plane is almost vertical. For example, when the surgeon unlock the saw 1240 to allow movement of the saw 1240 with respect to the robot arm to perform resection, the gravity force applied to the centers of gravity of the components of the end effector 1200, e.g., link segments 1210, 1220, may cause undesirable acceleration, which may lead to unexpected behavior of end effector 1200 structure. In addition to the risk of unintentional contact with the patient the saw 1240, the acceleration may tend to also apply a lateral force on the surgeon's hand depending on the orientation of the link segments 1210, 1220. For example, a configuration of the end effector 1200 can change suddenly without appropriate support, particularly when the link segments 1210, 1220 are substantially collinear (i.e., near a singularity configuration).

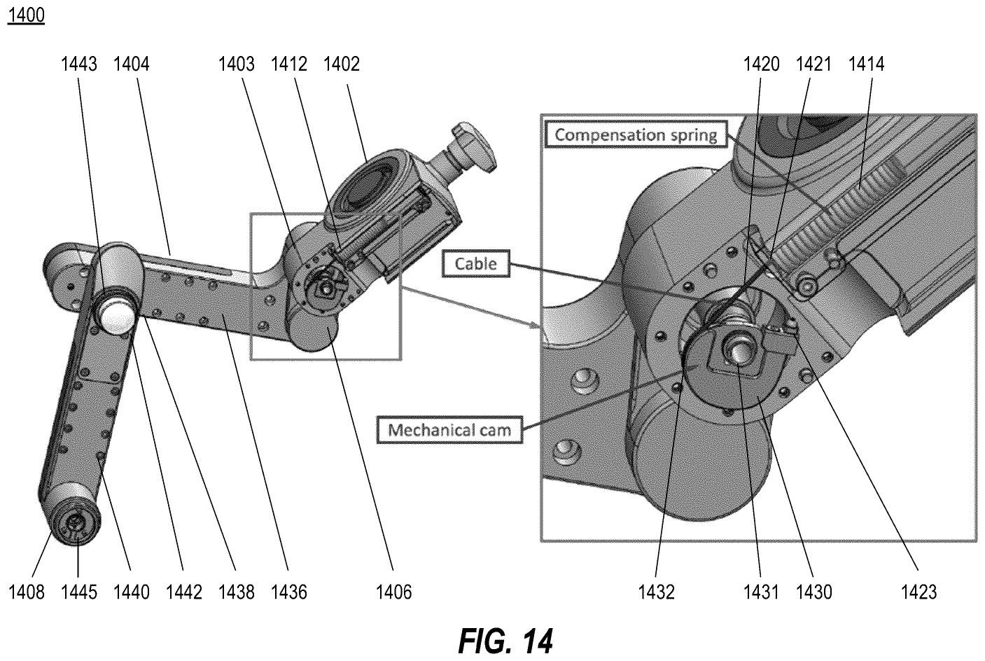

Thus, it is desirable to compensate for these undesirable forces on the end effector 1200 due to gravity when the end effector 1200 is in an inclined resection plane. In this regard, FIG. 14 illustrates an embodiment of an end effector arm 1400 having a spring mechanism 1412 for compensating for gravitational force on the end effector arm components, in accordance with some embodiments of the present disclosure. In this example, an end effector arm 1400 includes a base 1402 configured to attach to an end effector coupler of a surgical robot arm, such as the end effector coupler 22 of FIGS. 4 and 5 for example. The end effector arm 1400 includes a mechanical linkage 1404 including a first end 1406 rotatably coupled to the base 1402 at a base joint 1403 and a second end 1408 opposite the first end 1406. The second end 1408 is configured to be removably coupled to a handheld surgical tool, such as the saw 1240 of FIG. 12, for example. The end effector arm 1400 includes a spring mechanism 1412 configured to impart a variable rotational force on the mechanical linkage 1404 based on an angle of rotation of the mechanical linkage 1404 with respect to the base 1402.

As will be discussed below, this and other embodiments may compensate for the weight of the components of the end effector arm 1400, in order to avoid unexpected acceleration of the components when subjected to gravity, e.g., when the end effector arm 1400 is unlocked. These and other embodiments, may also reduce the effort and fatigue on a surgeon's hand due to the weight of the end effector arm 1400.

For example, the embodiment of example of FIG. 14 may be configured to fully compensate for the weight of the first link 1436 and partially compensate for the weight of the second link 1440 when the end effector arm 1400 is in a vertical resection plane. The end effector arm may also be configured to minimize variation of the compensation effect across the entire range of motion of the end effector arm 1400.

To achieve this and other advantages, a spring mechanism 1412 is integrated into the structure of the end effector arm 1400 and is configured to compensate for the weight of the end effector arm 1400 components without any electrical energy. For example, the spring mechanism 1412 in this embodiment provides elastic potential energy to compensate for a full loss of potential energy associated with the first link 1436 and a partial loss of potential energy associated with the second link 1440 when the resection plane is vertical.

In this example, a spring 1414 has a first end 1416 and a second end 1418, with the first end 1416 of the spring 1414 coupled to the base 1402 of the end effector arm 1400. A cable 1420 has a first end 1421 and a second end 1423, with the first end 1421 coupled to the first end 1416 of the spring 1414. In this example, spring 1414 is a compression coil spring disposed within a channel 1422 in the base 1402, with the first end 1416 of the spring 1414 abutting a stop 1428 in the channel and a boss 1426 disposed at the second end 1418 of the spring 1414. A guide rod 1424 is attached to the boss 1426, extends through the spring toward the first link 1436, and is attached to the first end 1421 of the cable 1420 such that tension in the cable 1420 is transferred to the second end 1418 of the spring 1414 to compress the spring 1414 and apply a counterforce on the cable 1420 based on the amount of compression in the spring 1414.

It should be understood, however, that other types of spring mechanisms and/or configurations may be used, as desired. For example, a suitable spring mechanism may use a tension coil spring, a hydraulic and/or pneumatic spring, elastomers, medical grade rubber bands, and/or any other suitable mechanism or components for applying a resilient counterforce in response to an applied force.

Referring back to FIG. 14, a cam 1430 is coupled to the first end 1406 of the mechanical linkage, e.g., via a shaft 1431 of the joint between the first link 1436 and the base 1402, such that the cam 1430 rotates with the first link 1436 with respect to the base 1402. The second end 1423 of the cable 1420 is coupled to the cam 1430, with the second end 1423 of the cable 1420 being received and guided within a cam guide 1432 of the cam 1430 during rotation of the mechanical linkage 1404 with respect to the base 1402.

It should be understood however, that other arrangements may be used, as desired. For example, in some embodiments, the spring may be disposed in the first link 1436, and the cam may be coupled to the base, such that the first link 1436 and spring rotate with respect to the base 1402 and the cam.

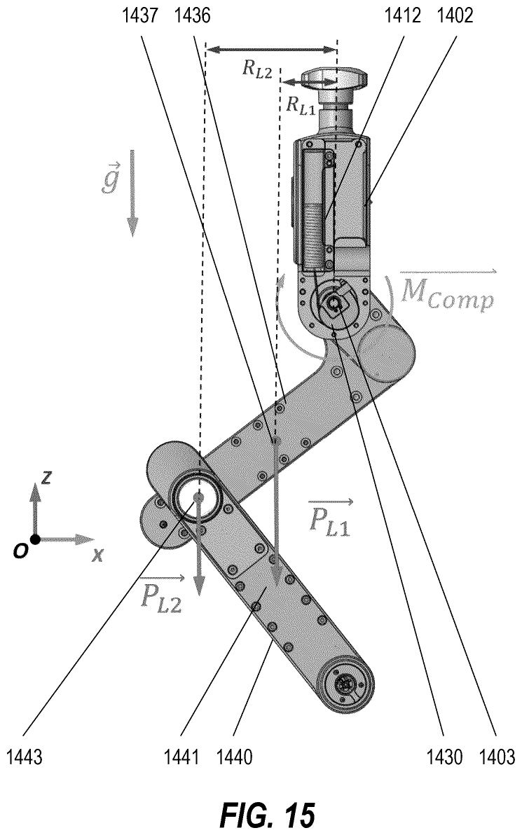

In this embodiment, the cam 1430 has a variable radius with respect to the shaft 1431, which causes rotation of the mechanical linkage 1404 with respect to the base 1402 at a linear rate to deform the spring 1414 at a non-linear rate. The cam 1430 may be configured such that, for each angular position of the first link 1436 with respect to the base 1402, a compensation torque M_{comp} is applied about the shaft 1431 that corresponds to an approximate moment generated by the gravity force applied to a center of gravity 1437 of the end effector arm 1400. The compensation torque M_{comp} may correspond to a sum of one or more components. For example, For example, as shown by FIG. 15, one component may be the amplitude of a moment about the base joint 1403 generated by the gravity force PL1 applied to the center of gravity 1437 of the first link 1436, with the moment M1 equal to PL1 times the horizontal distance RL1 between the center of gravity 1437 of the first link 1436 and the base joint 1403. Another component may be the amplitude of a moment generated by the gravity force PL2 corresponding to a defined fraction of the mass of the second link 1440 applied at the joint 1443 between the first end 1442 and the second end 1438 of the first link 1436, with the moment M2 equal to PL2 times the horizontal distance RL2 between joint 1443 and the base joint 1403. This is because the effective amplitude of the downward force PL2 may vary based on the vertical angle of the second link 1440, which in turn causes the actual distance of a center of gravity 1441 of the second link 1440 to vary as well. Thus, in this example, PL2 can be approximated based on any number of factors, such as an average amplitude of the actual gravitational force on the second link 240, a maximum amplitude, etc., as desired.

In some embodiments, the variable rotational force imparted by the spring mechanism 1412 is sufficient to overcome a gravitational force on the second end 1408 of the end effector arm 1400 through a range of angles of rotation of the mechanical linkage 1404 with respect to the base 1402. For example, the cam 1430 may be configured such that the variable rotational force is substantially equal to a gravitational force, an average gravitational force, and/or an approximate gravitational force on the second end 1408 of the end effector arm 1400 through the range of angles of rotation.

It should also be understood that multiple spring mechanisms may be employed in some embodiments. For example, in the example of FIGS. 14 and 15 above, a second spring mechanism may be added between the first link 1436 and the second link 1440 for example, to provide gravity compensation for the second link 1440 with respect to the first link 1436. This may permit the spring force of the primary spring mechanism 1412 to be reduced, as the total compensation torque being supplied by two mechanisms simultaneously. In some embodiments, a spring mechanism between the first link 1436 and the second link 1440 or between two other links, may be used in addition to, or in place of, the spring mechanism 1412, as desired.

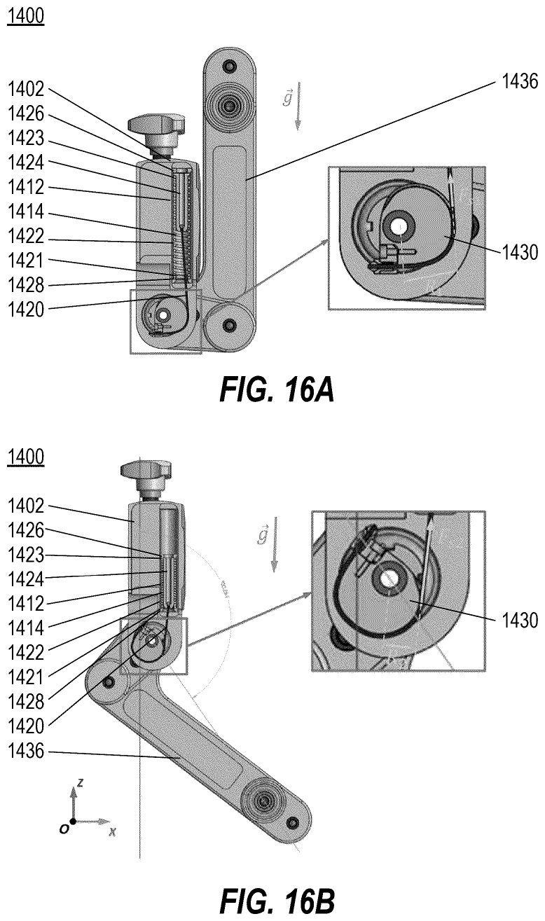

In this regard, FIGS. 16A and 16B illustrate a portion of the end effector arm 1400 of FIGS. 14 and 15 illustrating forces applied to the first link 1436 of the end effector arm 1400, according to some embodiments of the present disclosure. As shown by FIG. 16A, when the end effector arm 1400 is in a docked position, the center of gravity 1437 of the first link 1436 is horizontally offset from the vertical due to the shape of the first link 1436. In this example, the spring 1414 is configured to apply a tension force Fₛ₁ to the cable 1420, which imparts a compensation torque M_{comp1} equal to the tension force Fₛ₁ times the radius R₁ of the cam 1430 at the docked position angle. In this example, compensation torque M_{comp1} is set to be higher than the moment caused by the gravitational force on the mechanical linkage 1404, so that the mechanical linkage 1404 is biased toward the docked position, for safety, ergonomic, and/or other reasons, as desired.

As shown by FIG. 16B, the first link 1436 is at an angle of 142 degrees with respect to the docked position angle. The rotation of the cam 1430 causes the spring 1414 to compress by a length equal to the additional length of cable disposed in the cam guide 1432 due to the 142 degree rotation of the cam 1430. This compression of the spring 1414 proportionally increases the tension force Fₛ₂. Meanwhile, rotation of the mechanical linkage 1404 also varies the amount of torque needed to compensate for gravity on the mechanical linkage 1404 due to the change in the center of gravity of the mechanical linkage and in the horizontal distance of the center of gravity 1437 of the first link 1436 and/or mechanical linkage 1404 with respect to the base joint 1403. In this example, the radius R₂ of the cam 1430 at this angle is configured to produce a compensation torque M_{comp2}, equal to the tension force Fₛ₂ times the radius R₂, that is sufficient to overcome the gravitational force on the second end 1408 of the end effector arm 1400 for this angle. By customizing the profile of the cam 1430 and spring 1414, the compensation torque M_{comp} can be customized to compensate for the gravitational force on the mechanical linkage 1404 to minimize and/or eliminate the perceived weight of the mechanical linkage 1404 on the hand of a surgeon when the mechanical linkage 1404 is fixed in an angled resection plane.

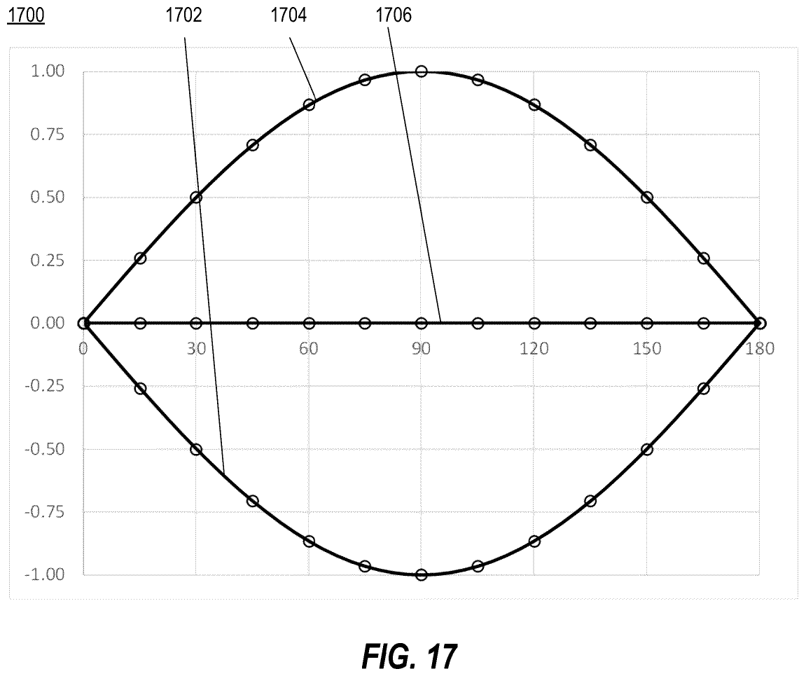

Referring now to FIG. 17, a graph 1700 is illustrated of the gravitational torque Mg 1702, the compensation torque M_{comp} 1704, and the net torque Mₙₑₜ 1706 on an end effector arm when the end effector arm is in a vertical resection plane, according to some embodiments of the present disclosure. In this example, the plot of gravitational torque M_{g} 1702 illustrates a torque applied at a base joint of the end effector arm due to gravitation force on the end effector arm between extension angles of 0 degrees (i.e., in a vertical docked position) and 180 degrees (i.e., in a fully extended position). In this example, the maximum torque due to gravitational force is at 90 degrees, when the horizontal distance between the center of gravity of the mechanical linkage and the base joint is greatest. In this example, the gravitational torque M_{g} 1702 can be expressed as:

$${M}_{g} = - {M}_{link} * {L}_{link} * ( sin θ ) * G$$

where Mₗᵢₙₖ is the mass of the mechanical linkage, Lₗᵢₙₖ is a distance from the center of mass of the mechanical linkage to the base joint, theta is the vertical angle of the line between the center of mass of the mechanical linkage and the base joint, and G is the gravitational constant.

Compensation torque M_{comp} 1704 can similarly be expressed as:

$${M}_{comp} = {M}_{link} * {L}_{link} * ( sin θ ) * G$$

By configuring the cam to produce this compensation torque M_{comp} profile across the sane range of angles, the net torque Mₙₑₜ 1706 can be customized to maintained at or near zero through the entire range of angles. For example, in the docked configuration of FIG. 16A, i.e., theta=0, M_{comp1} is relatively low due to the near-zero angle of the mechanical linkage 1404, and force Fₛ₁ is also relatively low due to the limited compression of the spring 1414, with the cam 1430 being configured with a radius R₁ to produce the desired M_{comp1}. As the mechanical linkage is rotated, M_{comp} increases to a maximum at 90 degrees with respect to vertical, with the profile of the cam 1430 being configured to take into account the cable interface point of the cam 1430 and a radius R to produce the appropriate compensation torque based on the increased spring force Fₛ. As shown by FIG. 16B, as the mechanical linkage 1404 approaches full stroke, i.e., 180 degrees, the compensation torque M_{comp} reduces, but the spring force Fₛ₂ continues to increase. By configuring the profile of the cam 1430 to reduce the radius R₂ as the cam 1430 rotates, this reduces the compensation torque M_{comp2} in two ways. First, the reduced radius offsets the increased spring force in the compensation torque equation. Second, the reduced radius reduces the rate of increase of the spring force as the cam 1430 rotates, thereby increasing the contribution of the reduced radius if the cam in the compensation torque equation at these larger angles.

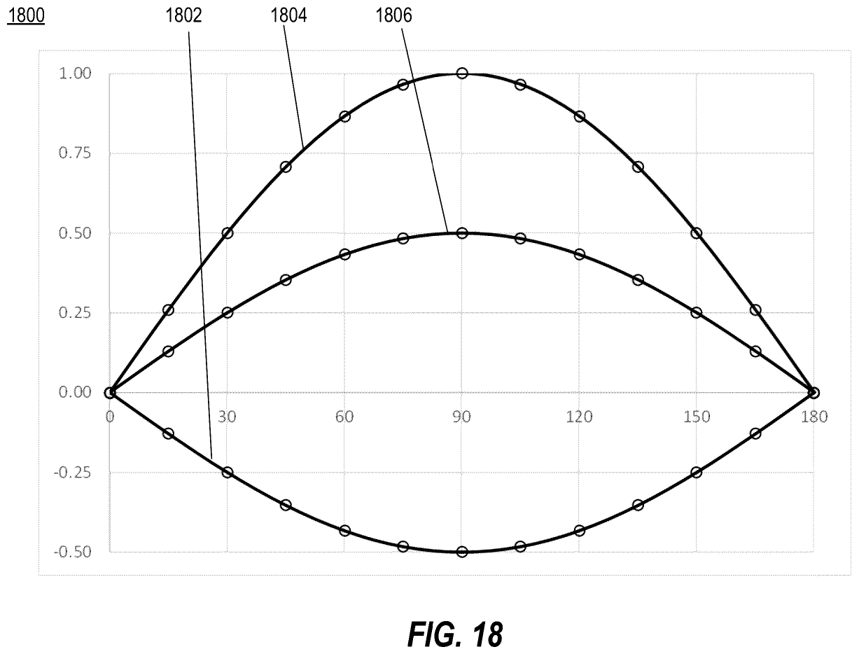

FIG. 18 is a graph 1800 of the gravitational torque M_{g} 1802, the compensation torque M_{comp} 1804, and the net torque Mₙₑₜ 1806 on an end effector arm when the end effector arm is in a resection plane of approximately 30 degrees with respect to horizontal, according to some embodiments of the present disclosure.

In this example, the gravitational torque M_{g} 1802 can be expressed as:

$${M}_{g} = - sin {θ}_{r} * {M}_{link} * {L}_{l i n k} ( sin {θ}_{link} ) G$$

where Mₗᵢₙₖ is the mass of the mechanical linkage, Lₗᵢₙₖ is a distance from the center of mass of the mechanical linkage to the base joint, theta is the vertical angle of the line between the center of mass of the mechanical linkage and the base joint, G is the gravitational constant, and Or is an angle of the resection plane with respect to horizontal. Thus, in this embodiment, the gravitational torque on the mechanical linkage in a 30 degree resection plane is approximately half of the corresponding gravitational torque in a vertical resection plane.

The equation for the compensation torque M_{comp} 1804 remains the same in this example:

$${M}_{c o m p} = {M}_{link} * {L}_{link} * ( sin θ ) * G$$

Thus, in this example, the net torque Mₙₑₜ 1706 can be expressed as

$${M}_{net} = M g + {M}_{c o m p} = ( 1 - sin {θ}_{r} ) * {M}_{link} * Llink * ( sin {θ}_{link} ) * G$$

Thus, in this example, the gravitational force is more than fully compensated by the spring mechanism of the end effector arm. In some examples, it may be preferable for the spring mechanism to cause the end effector arm to pull away from the patient rather than allow gravity to urge the end effector arm down toward the patient. The resulting net torque Mₙₑₜ may still be less than the corresponding gravitational torque, and a surgeon may find that pulling against the net torque to engage the surgical tool may be preferable to pushing the surgical tool upwards against the force of gravity during an operation.

As discussed above, FIGS. 19A-19C illustrate a locking mechanism 1906 configured to selectively fix a surgical tool 1904 with respect to the base 1902 of an end effector arm 1900. As shown in FIG. 19A, the tool 1904 includes a locking mechanism 1906 having a base 1908 that is fixed to the tool 1904. The base 1908 includes a channel 1910 for receiving a projection 1924 attached to a base 1902 of an end effector 1900 (see FIG. 19C). The locking mechanism 1906 also includes a spring-loaded locking block 1912 coupled to a pair of locking flanges 1914, each having a tapered engagement surface 1916. A manual actuator 1918 has a handle 1920 on either side of the actuator 1918.

As shown in FIG. 19B, a surgeon can press on a handle 1920 to move the actuator away from the base 1908, which causes the tapered engagement surfaces 1916 of the locking flanges 1914 to move away from an opposite interior wall 1922 of the channel 1910, thereby increasing an effective diameter of the channel 1910. As shown by FIG. 19C, the projection 1924 (e.g., a pin or other rigid structure) that is fixed to the end effector base 1902 of the end effector arm 1900 is inserted into the channel 1910 and the handle 1920 is released, which allows the spring loaded lock block to move the engagement surfaces 1916 of the locking flanges 1914 back toward the projection 1924 to clamp the projection 1924 between the tapered engagement surfaces 1916 of the flanges 1914 and the opposite interior wall 1922 of the channel 1910, thereby selectively fixing the surgical tool 1904 with respect to the end effector base 1902. The surgical tool 1904 can be selectively released from the end effector base 1902 by re-engaging the actuator 1918 to release the projection 1924, thereby allowing the surgical tool 1904 to be moved to remove the projection 1924 before re-releasing the actuator 1918.

### Further Definitions and Embodiments

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

We claim:

1. An end effector arm for use with a robotic surgical system comprising:
a base configured to attach to an end effector coupler of a surgical robot arm;
a mechanical linkage including a first end rotatably coupled to the base and a second end opposite the first end, the second end configured to be removably coupled to a handheld surgical tool; and
a spring mechanism configured to impart a variable rotational force on the mechanical linkage based on an angle of rotation of the mechanical linkage relative to the base so as to compensate for variable gravitational force on the mechanical linkage.

2. The end effector arm of claim 1, the spring mechanism comprising:
a spring including a first end and a second end, the first end of the spring coupled to one of the base and the first end of the mechanical linkage;
a cable including a first end and a second end, the first end of the cable coupled to the first end of the spring; and
a cam coupled to the other of the base and the first end of the mechanical linkage, the second end of the cable coupled to the cam, the cam including a cam guide configured to receive and maintain the second end of the cable within the cam guide during rotation of the mechanical linkage with respect to the base.

3. The end effector arm of claim 2, wherein rotation of the mechanical linkage in a first rotational direction with respect to the base at a linear rate causes the spring to deform at a non-linear rate.

4. The end effector arm of claim 2, wherein the spring comprises a compression coil spring.

5. The end effector arm of claim 2, wherein the spring comprises a tension coil spring.

6. The end effector arm of claim 2, wherein the spring comprises a hydraulic spring.

7. The end effector arm of claim 2, wherein the spring comprises a pneumatic spring.

8. The end effector arm of claim 1, wherein the variable rotational force is sufficient to overcome a gravitational force on the second end of the end effector arm through a range of angles of rotation of the mechanical linkage with respect to the base.

9. The end effector arm of claim 1, wherein the variable rotational force is substantially equal to a gravitational force on the second end of the end effector arm through a range of angles of rotation of the mechanical linkage with respect to the base.

10. The end effector arm of claim 1, the mechanical linkage comprising:
a first link including the first end of the mechanical linkage and a second end; and
a second link including the second end of the mechanical linkage and a first end rotatably coupled to the second end of the first link.

11. The end effector arm of claim 10, wherein the variable rotational force is sufficient to overcome a first gravitational force on the first link and at least a portion of a second gravitational force on the second link through a range of angles of rotation of the mechanical linkage with respect to the base.

12. The end effector arm of claim 1, further comprising a locking mechanism attachable to the surgical tool and configured to selectively lock the surgical tool with respect to the base.

13. A surgical tool guidance system comprising:
a robot arm configured to be positioned by a surgical robot, the robot arm including an end effector coupler;
an end effector arm including
a base configured to attach to the end effector coupler of the robot arm,
a mechanical linkage including a first end rotatably coupled to the base and a second end opposite the first end, and
a spring mechanism configured to impart a variable rotational force on the mechanical linkage based on an angle of rotation of the mechanical linkage relative to the base so as to compensate for variable gravitational force on the mechanical linkage; and
a handheld surgical tool configured to couple to the second end of the mechanical linkage.

14. The surgical tool guidance system of claim 13, the spring mechanism comprising:
a spring including a first end and a second end, the first end of the spring coupled to one of the base and the first end of the mechanical linkage;
a cable including a first end coupled to a first end of the spring and a second end; and
a cam coupled to the other of the base and the first end of the mechanical linkage, the second end of the cable coupled to the cam, the cam including a cam guide configured to receive and maintain the second end of the cable within the cam guide during rotation of the mechanical linkage with respect to the base.

15. The surgical tool guidance system of claim 14, wherein rotation of the mechanical linkage in a first rotational direction with respect to the base at a linear rate causes the spring to deform at a non-linear rate.

16. The surgical tool guidance system of claim 13, wherein the variable rotational force is sufficient to overcome a gravitational force on the second end of the end effector arm through a range of angles of rotation of the mechanical linkage with respect to the base.

17. The surgical tool guidance system of claim 13, the mechanical linkage comprising:
a first link including the first end of the mechanical linkage and a second end; and
a second link including the second end of the mechanical linkage and a first end rotatably coupled to the second end of the first link.

18. A surgical system comprising:
a tracking system configured to determine a pose of an anatomical structure that is to be engaged by a handheld surgical tool and to determine a pose of the surgical tool;
a surgical robot including
a robot base,
a robot arm connected to the robot base, the robot arm including an end effector coupler, and
at least one motor operatively connected to move the robot arm relative to the robot base,
an end effector arm including
a base configured to attach to the end effector coupler of the robot arm,
a mechanical linkage including a first end rotatably coupled to the base and a second end opposite the first end, and
a spring mechanism configured to impart a variable rotational force on the mechanical linkage based on an angle of rotation of the mechanical linkage relative to the base so as to compensate for variable gravitational force on the mechanical linkage; and
a handheld surgical tool coupled to the second end of the mechanical linkage.

19. The surgical system of claim 18, the spring mechanism comprising:
a spring including a first end and a second end, the first end of the spring coupled to one of the base and the first end of the mechanical linkage;
a cable including a first end coupled to a first end of the spring and a second end; and
a cam coupled to the other of the base and the first end of the mechanical linkage, the second end of the cable coupled to the cam, the cam including a cam guide configured to receive and maintain the second end of the cable within the cam guide during rotation of the mechanical linkage with respect to the base.

20. The surgical system of claim 19, wherein rotation of the mechanical linkage in a first rotational direction with respect to the base at a linear rate causes the spring to deform at a non-linear rate.
